(19) United States
(12) Patent Application Publication
(10) Pub. No.: US 2004/0241689 A1
(43) Pub. Date: Dec. 2, 2004

(54) Antigens of and antibodies to translocated molecules of microorganisms and uses thereof

(76) Inventors: Joel B. Baseman, San Antonio, TX (US); Thirumalai R. Kannan, San Antonio, TX (US); Rene Alvarez, San Antonio, TX (US)

Correspondence Address: MYERS BIGEL SIBLEY & SAJOVEC, PO BOX 37428, RALEIGH, NC, 27627, US

(21) Appl. No.: 10/485,002

(22) PCT Filed: Jul. 30, 2002
(86) PCT No.: PCT/US02/24121

Related U.S. Application Data
(60) Provisional application No. 60/309,128, filed on Jul. 31, 2001.

(51) Int. Cl. C12Q 1/68; A61K 39/00; A61K 39/38

(52) U.S. Cl. 435/6

(57) ABSTRACT

This application describes intracellular, cytoplasmic molecules that are translocated to the surface of a microorganism and participate in binding the microorganism to the surface of a host cell. Examples of such translocated molecules include glycerahdehyde 3-phosphate dehydrogenase, pyruvate dehydrogenase, and elongation factor-Tu. Regions of translocated molecules important for binding, as well as molecules which disrupt binding, are described. Antibodies directed to translocated molecules are also described.

This application is being filed as a PCT International Patent application ______ in the name of Board of Regents, University of Texas System, a U.S. national corporation, (applicant for all countries except the US, and Joel B. Baseman, Rene A. Alvarez, and T. R. Kannan, all residents and citizens of the U.S. (applicants for US only), on 30 Jul. 2002.

### STATEMENT REGARDING FEDERALLY SPONSORED RESEARCH OR DEVELOPMENT

The invention described herein was made under contract with the following agency of the United States Government: National Institutes of Health, NIH AI41010.

### FIELD OF THE INVENTION

The present disclosure relates to antigens of and antibodies to translocated molecules of microorganisms and compositions thereof. The disclosure also relates to methods of inhibiting binding of a microorganism to a surface of a host cell and methods of treating a disease in a subject due to an infection with a microorganism. The disclosure also relates to compounds and compositions for inhibiting the binding of a microorganism to the surface of a host cell.

### BACKGROUND OF THE INVENTION

Microorganisms are responsible for a great many diseases. The adherence of pathogenic microorganisms to host tissues is an important prerequisite for colonization and subsequent disease development.

Frequently, bacterial adherence to host tissues is mediated by a family of integrin receptors on host cell membrane surfaces. This interaction between bacteria and host cell surfaces triggers signal transduction pathways, which facilitate intracellular entry. While adhesins of bacterial pathogens, like Bordetella pertussis, possess RGD (arginine-glycine-aspartic acid) motifs that directly bind to integrins, other pathogens demonstrate alternate routes. For example, bacteria may interact directly with extracellular matrix (ECM) components such as collagen, laminin, keratin and fibronectin (Fn) in order to establish a focal point of infection or to target specialized tissue cells. In either case, bacteria express specific ligands to facilitate these interactions with integrins or ECM components.

Additional molecules have also been shown to mediate microbial adherence to extracellular matrix components of host cells. For example glyceraldehyde-3-phosphate dehydrogenase (GAPDH), a ‘cytoplasmic’ glycolytic enzyme has been described as a major surface protein in Saccharomyces cerevisiae and also as a FnBP in group A streptococci and Candida albicans. Additionally, anti-GAPDH enzymes have been shown to reduce the binding of C. albicans blastoconidia to fibronectin and laminin.

The inventors have surprisingly found that additional intracellular molecules translocated to the surface of a microorganism can mediate adhesion of a microorganism to the surface of a host cell.

### SUMMARY OF THE INVENTION

The present invention is directed to antigens of and antibodies to intracellular molecules translocated to the surface of microorganisms and uses thereof. The present invention includes identification and isolation of antigens of translocated molecules of microorganisms and antibodies to such antigens. The present invention is also directed to molecules capable of inhibiting binding of a microorganism to the surface of a host cell.

In one embodiment the invention provides a method for inhibiting the binding of a microorganism to a surface of a host cell or a molecule thereof. The method comprises contacting the microorganism with one or more antibodies to one or more translocated molecules, wherein the one or more translocated molecules is not GAPDH. The antibody can inhibit interaction between the surface of the host cell and the one or more transported intracellular molecule, thereby inhibiting the binding of the microorganism.

In one embodiment the invention provides a method for inhibiting the binding of a microorganism to a surface of a host cell or a molecule thereof. The method comprises contacting the microorganism with one or more antibodies to one or more translocated molecules, wherein the one or more translocated molecules includes a non-glycolytic enzyme.

In another embodiment the invention provides a method for inhibiting the binding of a microorganism to a surface of a host cell or a molecule thereof, the method comprises contacting the microorganism with one or more antibody to one or more translocated molecules, wherein the one or more translocated molecule comprises an anabolic enzyme.

In another embodiment the invention provides an isolated epitope of a translocated molecule of a microorganism, wherein the epitope is involved in binding the microorganism to a surface of a host cell or a molecule thereof. The epitope can comprise all or a portion of the molecule translocated to the surface of the microorganism. The epitope can be a fragment of the translocated molecule comprising a linear domain of the translocated molecule. The epitope can comprise a conformational-dependent domain, which may or may not constitute a linear domain of the molecule. The epitope can be linked to a carrier. In another embodiment, the invention provides an immunizing composition comprising an isolated epitope of a translocated molecule of a microorganism. In yet another embodiment, the invention provides antibodies to an isolated epitope of a translocated molecule of a microorganism.

In another embodiment, the invention provides a method for treating a disease in a subject due to an infection with a microorganism. The method comprises administering to the subject one or more antibodies to one or more translocated molecules of the microorganism, wherein the one or more antibodies inhibits binding between the surface of the host cell and the one or more translocated molecules.

In yet another embodiment, the invention provides a method for treating a disease in a subject due to an infection with a microorganism, the method comprising administering an immunizing composition to a subject, the immunizing composition comprising one or more antigens of one or more translocated molecules of a microorganism, wherein a humoral response to the antigen is produced, thereby producing one or more antibodies to the one or more translocated molecules.

In still another embodiment, the invention provides a method for treating a disease in a subject due to an infection with a microorganism, the method comprising administering a molecule to a subject, wherein the molecule inhibits binding of a translocated molecule of the microorganism to a surface of a cell of the subject.

In another embodiment, the invention provides compounds for inhibiting binding of a microorganism to the surface of a host cell. In one embodiment, the invention provides compounds that interact with a translocated molecule of a microorganism and interfere with the binding of the translocated molecule with the surface of a host cell or a molecule thereof. In another embodiment, the invention provides molecules that interact with the surface of a host cell or a molecule thereof to compete with binding of a translocated molecule of a microorganism with the host cell or molecule thereof. In another embodiment, the invention provides compounds that when contacted with a microorganism result in the internalization of a translocated molecule of the microorganism. In yet another embodiment, the invention provides compounds that directly or indirectly degrade a translocated molecule.

In another embodiment, the invention provides methods for inhibiting the binding of a microorganism or a translocated molecule thereof to the surface of a host cell or a molecule thereof. The method comprises contacting a microorganism or a translocated molecule thereof with one or more compounds; wherein the one or more compounds (a) interact with a translocated molecule of a microorganism and interfere with the binding of the translocated molecule with the surface of a host cell or a molecule thereof, (b) directly or indirectly cause the internalization of a translocated molecule of the microorganism; (c) directly or indirectly degrade a translocated molecule; or (d) a combination thereof.

In another embodiment the invention provides a method for treating a disease in a subject due to infection with a microorganism. The method comprises administering to the subject an effective amount of one or more compounds or pharmaceutically acceptable salts thereof, wherein the one or more compounds or pharmaceutically acceptable salts thereof (a) interact with a translocated molecule of a microorganism and interfere with the binding of the translocated molecule with the surface of a host cell or a molecule thereof, (b) compounds directly or indirectly cause the internalization of a translocated molecule of the microorganism; (c) directly or indirectly degrade a translocated molecule; or (d) a combination thereof.

In yet another embodiment, the invention provides compositions comprising one or more compound of the invention. The compositions can be pharmaceutical compositions.

### BRIEF DESCRIPTION OF THE DRAWINGS

FIG. 1 shows fibronectin (Fn)-binding proteins of M. pneumoniae. Total mycoplasma protein lysates were separated by 12% SDS-PAGE and transferred to nitrocellulose membranes, which were incubated with or without 20 μg/ml of human plasma Fn followed by rabbit anti-Fn antisera (1:1000 in blotto). Peroxidase-conjugated goat anti-rabbit Abs (1:3000 in 1% blotto) were added and color developed. Positions of the 30- and 45-kDa proteins are indicated to the left. The high molecular band, which appeared in both lanes, was considered non-specific binding.

FIG. 2 compares Fn-binding activity of wild-type HA⁺ M. pneumoniae and class II HA⁻ mutant of M. pneumoniae lacking P30 adhesin. Total M. pneumoniae protein lysates were separated by 12% SDS-PAGE and transferred to nitrocellulose membranes. (A): probed for FnBPs using Fn and anti-Fn Abs; (B): probed for P30 adhesin using anti-P30 adhesin Abs (1:1000 in 1% blotto). Wt, wild-type: HA⁻, hemadsorption-negative. Molecular weight markers are indicated to the left (from top: 215, 99, 71, 44, 28, 19, and 14-kDa). The 30- and 45-kDa protein positions are marked.

FIG. 3 shows SDS-PAGE autoradiograph of M. pneumoniae proteins eluted from control and Fn-Sepharose affinity columns. Mycoplasma protein lysates were incubated with Fn-coupled Sepharose on a platform rocker at 4° C. for 24 h. Affinity columns were prepared and washed extensively to remove unbound proteins. Bound proteins were eluted with 5M LiCl, and eluted fractions with high radioactivity were processed by Amicon concentration. Specific fractions were resolved using 12% SDS-PAGE gels, electrophoretically transferred to PVDF membranes and exposed to X-ray film. Positions of the 30- and 45-kDa FnBPs are indicated to the right.

FIG. 4 shows a complete amino acid sequences of M. pneumoniae FnBPs, (A) EF-TU (SEQ ID NO: 1) and (B) PDH-B (SEQ ID NO: 2). NH₂-terminal sequences of the Fn-Sepharose column-purified proteins are typed in bold letters. Boxed amino acid sequences are the NH₂-terminal sequences of the same proteins obtained from SDS-PAGE gels. The amino acid tryptophan (W) is indicated in bold, and the tryptophan coded by UGA at amino acid position 245 in PDH-B is also underlined.

FIGS. 5A-C show an analysis of recombinant M. pneumoniae FnBPs. (A) I. Overexpressed and column-purified EF-Tu protein from construct pET-EF-Tu. (A) II. Immunoblots of rabbit prebleed serum and anti-rEF-Tu antiserum against total M. pneumoniae proteins. (B) I. Overexpressed and purified rNPDH from construct pET-NPDH (amino acids 1-244 of PDH-B). (B) II. Immunoblots of rabbit prebleed serum and anti-rNPDH antiserum against total M. pneumoniae proteins. Molecular weight markers are indicated to the left. (C) Fn binding of recombinant FnBPs using ligand immunoblot assay. Purified rEF-Tu and rNPDH-B proteins were separated by 12% SDS-PAGE, transferred to nitrocellulose membranes and probed with Fn and anti-Fn Abs as described in the brief description of FIG. 1.

FIG. 6 shows binding of recombinant EF-Tu and PDH to immobilized fibronectin (Fn). Microtiter wells were coated with 100 ng of human Fn. Increasing concentrations of rEF-Tu (□) and rNPHH (Δ) were incubated in individual wells for 1 h at room temperature. Bound protein was detected with anti-rEF-Tu or anti-rNPDH polyclonal antibodies and goat anti-rabbit alkaline phosphatase-conjugated polyclonal antibodies, followed by p-nitrophenyl phosphate substrate. Values represent the means of triplicate wells from 3 separate experiments. rNPDH-B refers to recombinant N-terminal PDH-B, which is ⅚^{th} of the entire PDH-B protein from the N-terminal domain expressed in E. coli.

FIG. 7 shows immunogold electron microscopy detection of EF-Tu and PDH-B proteins on M. pneumoniae cell surfaces. Mycoplasmas were incubated with antisera (1/100) generated against rEF-Tu and/or r-NPDH-B and rabbit IgG (1/20) gold particles (size 10 nm or 20 nm). Gold labeling of PDH-B (panel A, 10 nm) showed both membrane and tip-associated localization. In contrast, gold labeling of EF-TU (panel B, 20 μm) revealed random membrane distribution. Furthermore, gold particle double labeling (panel C) confirmed the contrasting distribution of PDH-B (10 nm) and EF-Tu (20 μm) on the M. pneumoniae membrane and tip surfaces (Bar=0.1 μm).

FIG. 8 shows surface location of M. pneumoniae FnBPs. Whole cell radioimmunoprecipitation (WCRIP) was performed using [³⁵S]-methionine biosynthetically-labeled viable M. pneumoniae reacted with rabbit prebleed or immune sera generated against overexpressed rEF-Tu and rNPDH. Total [³⁵]-methionine labeled M. pneumoniae proteins were separated by 12% SDS-PAGE. Positions of the 30- and 45-kDa proteins are indicated to the right.

FIG. 9 shows dose-dependent binding of M. pneumoniae to immobilized Fn. Microtitre plates were coated with varying concentrations of human plasma Fn (0.001-10 μg/well). Individual wells were washed, and unoccupied sites were blocked with 1 mg/ml BSA. [³⁵S]-methionine-labeled viable mycoplasmas were added to each well, non-adherent cells were removed by washing, and radioactivity was counted. Wells with BSA (1 mg/ml) alone served as negative controls.

FIG. 10 shows inhibition of M. pneumoniae binding to Fn. [³⁵S]-methionine biosynthetically-labeled viable M. pneumoniae cells were preincubated for 1 h with 1:100 dilutions of prebleed sera or antisera generated against rFnBPs, prior to adding mycoplasmas to microtitre wells containing immobilized Fn at 0.1 μg/well.

FIG. 11 shows binding of M. genitalium to mucin-coated surfaces. A) ELISA plates were coated with 0.02-20 Δg of purified human vaginal/cervical (V/C) mucin. ³⁵S radiolabeled, viable mycoplasmas were added to the individual wells and incubated at 37° C. for 1 hr. Plates were rinsed with PBS and radioactivity was measured. B) Plates were coated with 2 mg of human V/C mucin, and radiolabeled mycoplasmas were pretreated with 0 to 10 μg of mucin prior to incubation with mucin-coated plates. Plates were rinsed with PBS, and radioactivity was measured. C) Plates were coated with 2 μg of human V/C mucin, and radiolabeled mycoplasmas were pretreated with 10 to 100 mM quantities of each mucin-associated sugar prior to addition to mucin-coated plates. Rhamnose and mannose were used as negative controls. Plates were rinsed and radioactivity was determined.

FIG. 12. Identification of M. genitalium mucin-binding proteins. A) M. genitalium ³⁵S Met biosynthetically labeled lysate was passed through a mucin-epoxy affinity column. Three proteins of 36, 38 and 40 kDa were eluted with 2.5 M LiCl. B) The purified 38 kDa protein N-terminal sequence (blue) had 100% homology with glyceraldehyde 3-phosphate dehydrogenase (GAPDH).

FIG. 13. Characterization of M. genitalium rGAPDH. A) rGAPDH was overexpressed in E. coli BL21 [DE3] and purified by nickel chromatography. B) Lane 1 represents whole cell lysates of M. genitalium reacted with a 1/1000 dilution of anti-rGAPDH rabbit serum. Lane 2 represents the same preparation reacted with pre-immune serum.

FIG. 14. Comparison of mucin binding activity of M. genitalium in the presence of pre-immune and anti-rGAPDH rabbit sera. A 67% reduction in binding activity occurred when radiolabeled mycoplasmas were pretreated with anti-rGAPDH before incubating with immobilized mucin on ELISA plates. No reduction in binding was observed when radiolabeled mycoplasmas were pretreated with anti-P140 and anti-P32 M. genitalium adhesin antibodies before incubating with immobilized mucin on ELISA plates.

FIG. 15. Whole cell radioimmumoprecipitation of GAPDH with and without trypsin treatment. M. genitalium whole cells were biosynthetically labeled with ³⁵S Met and treated with pre-immune (PS) or anti-rGAPDH sera. Cells were lysed, and M. genitalium protein immune complexes were precipitated with protein A. Whole cells were also treated with trypsin prior to immunoprecipitation. Mycoplasma were also separated by SDS-PAGE.

FIG. 16. Immunoelectron microscopy of M. genitalium labeled with polyclonal serum against GAPDH. M. genitalium whole cells were washed and treated with anti-rGAPDH diluted sera (1:100) and goat anti-rabbit IgG-gold complex (10 nm particle size). Mycoplasma were washed, fixed, and stained with 7% uranyl acetate and examined with a transmission electron microscope for the presence of GAPDH-gold particle complexes on M. genitalium.

FIG. 17 is a graph showing the inhibition of Mycoplasma genitalium binding to mucin by antibodies against GAPDH and PDH-B.

FIG. 18 is a representation of complete GAPDH and truncated fragments thereof used in mucin binding studies.

FIG. 19 is a graph showing binding of M. genitalium to mucin-coated surfaces. Plates were coated with 2 μg of human vaginal/cervical (V/C) mucin, porcine gastrointestinal (GI) mucin, bovine submaxillary mucin (BSI) and bovine serum albumin (BSA). Radiolabeled mycoplasmas were then added to each well. Plates were rinsed with PBS and radioactivity determined.

FIG. 20 is a graph showing results of a competitive inhibition of Mycoplasma pneumoniae binding with fibronectin (Fn) by recombinant Fn binding proteins. Microtitre plate wells were coated with 100 ng of Fn for 16 h at 4° C. The Fn coated wells were then preincubated with different concentrations of recombinant EF-Tu, PDH-B and EF-Tu/PDH-B in combination at 37° C. for 2 h. Individual wells with Fn and M. pneumoniae served as positive control. Individual wells with BSA and M. pneumoniae served as negative control.

### DETAILED DESCRIPTION

Overview

The present invention is directed to antigens of and antibodies to translocated molecules of microorganisms and uses thereof. As used herein, “translocated molecule” means a molecule that typically performs a function in the cytosol and is either actively or passively transported to the extracellular surface of the cell. The antigens and antibodies of the invention, as well as other molecules described herein, can be used to inhibit binding of a microorganism to a surface of a host cell or molecules thereof.

Binding of a microorganism to a host cell is typically mediated through interaction of specialized molecules on the surface of the microorganism and molecules on the surface of the host cell. It is disclosed herein that molecules typically considered cytosolic can be translocated to the surface of microorganisms and can be involved in binding the microorganism to the surface of a host cell. Examples of molecules typically considered cytosolic include, for example, nucleotides, nuclear receptors, anabolic enzymes, and glycolytic and non-glycolytic enzymes, and fragments thereof. In one embodiment of the invention, antibodies to translocated non-glycolytic enzymes of a microorganism can be used to inhibit binding of a microorganism to a surface of a host cell or molecules thereof. In another embodiment, antibodies to translocated anabolic enzymes of a microorganism can be used to inhibit binding of a microorganism to a surface of a host cell or molecules thereof.

In another embodiment of the invention, antibodies to glycolytic enzymes can be used to inhibit binding of a microorganism to a surface of a host cell or molecules thereof. Glycolytic enzymes perform well-defined functions within the cytosol. Glycolytic enzymes are enzymes involved in the glycolytic pathway and are responsible for a major source of energy for cells. Because of the metabolic nature of glycolytic enzymes, it is surprising that glycolytic enzymes can be transported to the surface of a microorganism and be involved in binding the host cell. In one embodiment of the invention, antigens can include antigens of newly identified translocated glycolytic enzymes of a microorganism.

In another embodiment, the invention provides a method for inhibiting the binding of a microorganism to the surface of a host cell through the use of antibodies to translocated glycolytic enzymes of microorganisms, with the proviso that the enzyme is not GAPDH.

In another embodiment, the invention provides a method for inhibiting the binding of a microorganism to the surface of a host cell or a molecule thereof including contacting the microorganism with an antibody to a translocated molecule of the microorganism, wherein the antibody inhibits the binding of the translocated molecule to mucin on the surface of the host cell.

In yet another embodiment, the invention provides a method for inhibiting the binding of a microorganism to the surface of a host cell or a molecule thereof including contacting the microorganism with an antibody to a translocated molecule of the microorganism, wherein the antibody inhibits the binding of the translocated molecule to fibronectin on the surface of the host cell.

In another embodiment of the invention, isolated antibodies to translocated molecules of a microorganism involved in the binding of a surface of a host cell or molecules thereof are provided. In yet another embodiment, the invention provides antibodies to epitopes of tranlocated molecules of a microorganism, wherein the epitopes are involved in binding a microorganism to a host cell surface. In another embodiment the invention provides the use of antibodies to epitopes of tranlocated molecules of a microorganism to inhibit the binding of a microorganism to the surface of a host cell.

Other embodiments not explicitly recited herein will become evident in light of the disclosure.

Abbreviations

The following is a list of abbreviations and their corresponding definitions as will be used throughout the specification. Any other abbreviations used will be understood from the context in which they are used.

Ab: Antibody

BSA: Bovine serum albumin

BSI mucin: Bovine submaxillary type I mucin

DMEM: Dulbecco's minimal essential medium

ECM: Extracellular matrix

EF-Tu: Elongation factor-Tu

ELISA: Enzyme linked immunosorbent Assay

Fn: Fibronectin

FnBPs: Fibronectin binding proteins

GAPDH: Glyceraldehyde-3-phosphate dehydrogenase

HA: Hemadsorption

HA⁻: Hemadsorption negative

HA⁺: Hemadsorption positive

HAT: hypoxanthine-aminopterin-thymidine

HSA: Human serum albumin

KLH: Keyhole limpet hemocyanin

LB broth: Luria Bertani broth

NPDH: Pyruvate dehydrogenase E1 □ subunit from amino acid 1 to 244

ORF: Open reading frame

PAGE: Polyacrylamide gel electrophoresis

PBS: Phosphate buffered saline

PBST: Phosphate buffered saline containing 0.05% Tween-20

PDH-A: Pyruvate dehydrogenase E1 □ subunit

PDH-B: Pyruvate dehydrogenase E1 □ subunit

rEF-Tu: recombinant EF-Tu

rGAPDH: Recombinant GAPDH

RGD: Arginine-glycine-aspartic acid

RIP: Radioimmuno precipitation

rNPDH: recombinant NPDH

SDS: Sodium docecyl sulfate

SPDP: N-succinimidyl-3-(2-pyridyldithio) proprionate

SRBC: Sheep erythrocytes

TDSET: 10 mM Tris [pH 7.8], 0.2% sodium deoxycholate, 0.1% SDS, 10 mM tetrasodium EDTA, 1% Triton X-100

WCRIP: Whole Cell Radio Immuno Precipitation

Definitions

All scientific and technical terms used in this application have meanings commonly used in the art unless otherwise specified. As used in this application, the following words or phrases have the meanings specified.

As used herein, “translocated molecule” means a molecule that typically performs a function in the cytosol and is either actively or passively transported to the extracellular surface of the cell. Transported molecules can include carbohydrates, proteins, lipids, nucleic acids, and combinations and fragments thereof. Functions performed by translocated molecules can include providing templates and building blocks for macromolecular synthesis, catalyzing reactions involved in macromolecular synthesis, providing structural support or integrity for a cell, catalyzing reactions involved in producing energy, and transporting molecules throughout the cell. Transported molecules can include, nucleotides, nuclear receptors, anabolic and catabolic enzymes, and glycolytic and non-glycolytic enzymes. An example of an anabolic and nonglycolytic enzyme that can be a translocated molecule is EF-Tu. An example of a glycolytic enzyme that can be a translocated molecule is pyruvate dehydrogenase.

Translocated molecules can include peptides, including enzymes, having a transmembrane domain. Transmembrane domains are typically composed of one or more stretches of 10-30 predominantly hydrophobic amino acid residues. Often the hydrophobic residues are separated by polar connecting loops. The stretches of about 10-30 amino acids often form α-helical segments, but can also form β-barrel segments. To determine whether a peptide contains a transmembrane domain, its primary amino acid sequence can be compared with an amino acid sequence of a known transmembrane domain.

As used herein, “surface” refers to the extracellular portions of a cell that are accessible to molecules outside of and apart from the cell. Surface can include cell wall and plasma membrane, extracellular molecules or portions thereof at least partially imbedded in the cell wall and plasma membrane, and extracellular molecules or portions thereof associated with the cell wall and plasma membrane. When a cell does not contain a cell wall, surface refers to plasma membrane, extracellular molecules or portions thereof at least partially imbedded in the plasma membrane, and extracellular molecules or portions thereof associated with plasma membrane.

As used herein, “metabolic” describes a chemical change in living cells by which energy is provided for vital processes and activities and new material is assimilated.

As used herein, “anabolic enzyme” refers to an enzyme involved in the constructive part of metabolism concerned especially with macromolecular synthesis. Anabolic enzymes include enzymes involved in oligonucleotide synthesis, oligosaccharide synthesis; lipid synthesis; and polypeptide synthesis. Anabolic enzymes include enzymes that catalyze condensation reactions directly resulting in the production of macromolecules, e.g., the formation of a peptide bond, and include enzymes, e.g., EF-Tu, responsible for orienting molecules, e.g. amino acids, in a position such that condensation reactions can occur. Anabolic enzymes include enzymes that typically catalyze reactions that involve the hydrolysis of GTP to GDP. As used herein, anabolic enzyme also refers a portion of a full enzyme, whether the portion is active or inactive with regard to its anabolic function.

As used herein, “catabolic enzyme” refers to an enzyme involved in the overall process of destructive metabolism involving the release of energy and resulting in the breakdown of complex materials within the organism. Some catabolic enzymes, for example some glycolytic enzymes, catalyze reactions that result in a net use of energy. As used herein, catabolic enzyme also refers to a portion of a full enzyme, whether the portion is active or inactive with regard to its catabolic function.

As used herein, “glycolytic enzyme” refers to an enzyme that is involved in the glycolytic pathway. The glycolytic pathway refers to overall process of the enzymatic breakdown of a carbohydrate with a resultant production of energy. Some glycolytic enzymes catalyze reactions that result in a net use of energy. Glycolytic enzymes include, for example, glyceraldehyde-3-phosphate dehydrogenase (GAPDH), enolase, phosphoglycerate kinase, alcohol dehydrogenase, pyruvate kinase, and aldolase. As used herein, “non-glycolytic enzyme” refers to an enzyme that is not involved in the glycolytic pathway. As used herein, glycolytic enzyme also refers to a portion of a full enzyme, whether the portion is active or inactive with regard to its glycolytic function.

As used herein, “treating” or “treatment” means the prevention or reduction of severity of symptoms or effect of a pathological condition. In referring to an infection with a microorganism, treating or treatment includes reducing the number of the microorganisms within a subject or preventing or reducing the severity of symptoms. Treating can also include prolonging life expectancy of a subject.

As used herein, “binding” means an interaction between two molecules such that energy is required to break up the interaction. Binding interactions typically include hydrogen binding and van der Waal's interactions. Binding includes the interaction of a microorganism and the surface of a host cell or a molecule thereof. Binding also includes the interaction of a molecule on the surface of a microorganism and a molecule of the surface of a host cell. Inhibition of binding refers to preventing or decreasing binding. Referring to binding between a microorganism and the surface of a host cell or a molecule thereof, the inhibition of binding can be measured by comparing the number of microorganisms bound to the surface of host cells or molecules thereof before, for example, contacting the microorganisms with an antibody to a translocated molecule to the number of microorganisms bound after contacting the microorganism with the antibody. Changes in binding affinity can also be used to detect inhibition of binding.

As used herein, “antigen” refers to a molecule to which an antibody binds. Antigens can be included in immunizing compositions. An antigen of the invention can be a translocated molecule of a microorganism or a fragment thereof. An antigen of the invention may or may not be purified from the surface of a microorganism.

As used herein, “epitope” refers to that portion of an antigen that immunoreacts with an antibody. An epitope of the invention can include all or a portion of the molecule translocated to the surface of the microorganism. An epitope can be a fragment of the translocated molecule including a linear domain of the translocated molecule. An epitope can include a conformational-dependent domain, which may or may not constitute a linear domain of the molecule. An epitope of the invention can be linked to a carrier. As used herein, “isolated epitope” is used interchangeably with “isolated antigen.”

As used herein, an “immunizing composition” is a composition which when administered to an animal stimulates production of antibodies that react with an antigen present in the immunzing composition. An immunizing composition of the invention can also be used as a vaccine for treatment or prevention of an infection with a microorganism.

As used herein, the term “isolate” means that an entity is in an environment other than that found in nature. When referring to an antigen, means that the antigen is separated from an affected subject in a form suitable for identification or for use in an immunizing or therapeutic composition, with or without further purification.

As used herein, “antibody” refers to a protein functionally defined as a binding protein and structurally defined as including an amino acid sequence that is recognized as being derived from the framework region of an immunoglobulin encoding gene of an animal producing antibodies. Antibodies can be intact immunoglobulins or fragments thereof, including single chain Fv, Fv, Fab, disulfide linked Fv, and F(ab)′₂. Antibodies can be monoclonal or polyclonal.

As used herein, “microorganism” means a single cell organism capable of growth and reproduction outside of living host cells. Microorganism includes mycoplasma, bacteria, and yeast. Mycoplasma, bacteria, and yeast can be pathogenic or non-pathogenic.

A “host cell” according to the invention is any cell to which a microorganism can bind. The host cell can be, for example, a plant cell or a mammalian cell. Mammalian cells can be cells from, for example, mice, rats, pigs, chickens, horses, cats, dogs, elephants, giraffes, monkeys, or humans, and the like.

A “subject” according to the invention includes any multicellular organism that can be infected with a microorganism. For example, a subject can be a plant or a mammal. Mammalian subjects include, for example, mice, rats, pigs, chickens, horses, cats, dogs, elephants, giraffes, monkeys, or humans, and the like.

As used herein, “mucin” refers to family of glycoprotein of the thick gelatinous layer of mucosal epithelium generally known as mucin or mucins and includes mucin-like glycoproteins. An example of mucin is bovine submaxillary type I mucin.

As used herein, “EF-Tu” functionally refers to a polypeptide that delivers an aminoacyl-tRNA complimentary to a nucleotide of an mRNA template to the A site (acylation site) of a ribosome. “EF-Tu” structurally refers to a polypeptide encoded by an oligonucletide having the sequence of SEQ ID NO: 1, an oligonucleotide capable of hybridizing to an oligonucleotide of SEQ ID NO: 1 under stringent conditions, or an oligonucleotide having about at least 80% sequence identity to an oligonucleotide of SEQ ID NO: 1. EF-Tu also refers to polypeptide encoded by an oligonucletide having about at least 85%, 90%, 95%, or 99% sequence identity to an oligonucleotide of SEQ ID NO: 1.

As used herein, “pyruvate dehydrogensae” refers to an enzyme that catalyzes the oxidative decarboxylation of pyruvate. Pyruvate dehydrogenase E1 alpha (PDH-A) and pyruvate dehydrogenase E1 beta (PDH-B) are examples of such enzymes. PDH-A or PDH-B can form a pyruvate dehydrogenase component of a pyruvate dehydrogenase complex. A pyruvate dehydrogenase complex includes a pyruvate dehydrogenase (E1) component, a dihydrolipoyl transacetylase (E2) component, and a dihydrolipoyl dehydrogenase (E3) component.

As used herein, “pyruvate dehydrogenase E1 beta (PDH-B)” refers to a polypeptide encoded by an oligonucletide having the sequence of SEQ ID NO: 2, an oligonucleotide capable of hybridizing to an oligonucleotide of SEQ ID NO: 2 under stringent conditions, or an oligonucleotide having about at least 80% sequence identity to an oligonucleotide of SEQ ID NO: 2. PDH-B also refers to polypeptide encoded by an oligonucletide having about at least 85%, 90%, 95%, or 99% sequence identity to an oligonucleotide of SEQ ID NO: 2.

“Percent (%) sequence identity” means the percentage of nucleotide residues in a particular oligonucleotide sequence (sequence A) that are identical with nucleotides in another sequence (sequence B), after aligning the sequence and introducing gaps, if necessary to achieve the maximum sequence identity. Known sequence comparison software can be used to determine percent sequence identity. For example, sequence comparison can be preformed using the program NCBI-BLAST2 (Altschul et al., Nucleic Acids Res. 25:3389-3402 (1997)). The NCBI-BLAST2 sequence comparison program may be downloaded from http://www.ncbi.nlm.nih.gov.

“Stringency” of hybridization reactions is readily determinable, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Host Cell Surface

In one embodiment, the invention provides a method for inhibiting the binding of a microorganism to the surface of a host cell or a molecule thereof by contacting the microorganism with an antibody to one or more translocated molecule of the microorganism. The use of antibodies to translocated molecules of a microorganism to prevent binding of the translocated molecule to the plasma membrane, extracellular portions of molecules at least partially imbedded in the plasma membrane, or extracellular molecules associated with the plasma membrane or extracellular portions of molecules at least partially imbedded in the plasma membrane of a host cell are contemplated in the present invention. The use of antibodies to translocated molecules of a microorganism to prevent binding to at least partially purified molecules of the surface of a host cell is also contemplated.

It is contemplated that antibodies to one or more translocated molecules of a microorganism that bind one or more cell adhesion molecules, integrins, extracellular matrix molecules, or glycoproteins of the thick gelatinous layer of mucosal epithelium of a host will be used to inhibit binding of the microorganism to the surface of a host cell or to an at least partially purified cell adhesion molecule, integrin, extracellular matrix molecule, or glycoprotein of the thick gelatinous layer of mucosal epithelium. Extracellular matrix molecules to which the one or more translocated molecule of the microorganism bind include extracelluar matrix proteins such as collagens, proteoglycans, elastin, and fibronectin, vitronectin, thrombospondin, decorin, heparin sulfate and laminin.

In one embodiment, the invention provides antibodies to antigens of translocated molecules of microorganisms that bind mucin, which is a glycoprotein of the thick gelatinous layer of mucosal epithelium.

Translocated Molecules

In one embodiment, the invention provides antigens of translocated molecules of a microorganism that are capable of binding the surface of a host cell. In another embodiment, the invention provides antibodies to the antigens.

Translocated molecules can include carbohydrates, proteins, lipids, nucleic acids, and combinations and fragments thereof. Preferably, the antigens of the transported molecules or fragments thereof are capable of producing an immunogenic response when included in an immunizing composition as described herein. Translocated molecules of a microorganism can include enzymes. The enzymes can be anabolic, catabolic, glycolytic or non-glycolytic. In one embodiment, the translocated enzymes of a microorganism include anabolic and/or non-glycolytic enzymes, or fragments thereof. EF-Tu serves as an example of both a non-glycolytic and an anabolic enzyme useful in the present invention. In another embodiment, the translocated enzymes include glycolytic enzymes. Pyruvate dehydrogenase serves as examples of such an enzyme.

Mycoplasmas

In one embodiment, the invention provides a method for inhibiting the binding of mycoplasma to the surface of a host cell or a molecule thereof. Mycoplasmas are the smallest organisms known capable of growth and reproduction outside of living host cells. Unlike other prokaryotic cells and yeast, mycoplasmas lack a cell wall. Mycoplasmas do not synthesize cell wall components, such as muramic acid or diaminopimelic acid. These and other differences between mycoplasma and other prokaryotic organisms and yeast are known.

Mycoplasmas are known to infect and cause diseases in animals and humans and plants. For example, infection with mycoplasmas has been shown to cause pleuropneumonia in cattle, arthritis in rats, and neurologic disorders of mice, such as rolling disease. Mycoplasma have also been shown to be the causative agent of primary atypical pneumonia in humans. They have also been isolated from the joints of humans with arthritis and have been associated with inflammation of the human genito-urinary tract. These and other diseases associated with mycoplasmas are known and are contemplated to be treated by the methods of the present invention. The invention provides methods for treating disease in a subject due to infection with a mycoplasma.

In another embodiment, the invention provides a method for inhibiting the binding of a M. genitalium to the surface of a host cell or a molecule thereof. Mycoplasma genitalium is responsible for human urethritis and implicated in pneumonia and arthritides (Baseman, Subcell Biochem 20: 243-59, 1993; Baseman et al., Isr J Med Sci 20: 866-9, 1984; Baseman et al., Microb Pathog 19: 105-16, 1995; Baseman and Tully, Emerg Infect Dis 3: 21-32, 1997; Giron et al., Infect Immun 64: 197-208, 1996). These and other diseases associated with M. genitalium are known and are contemplated to be treated by the methods of the present invention.

In another embodiment, the invention provides a method for inhibiting the binding of a M. pneumoniae to the surface of a host cell or a molecule thereof. Mycoplasma pneumoniae is a human bacterial pathogen that causes tracheobronchitis and primary atypical pneumonia. Furthermore, M. pneumoniae can disseminate to other organ sites and cause gastrointestinal, hematologic, neurologic, dermatologic, musculoskeletal and cardiovascular pathologies (Baseman et al., 1996). This secondary involvement by M. pneumoniae leads to a spectrum of complicated sequelae, including asthma, arthritis, pericarditis, and central nervous system disorders, which attests to the significance of M. pneumoniae in human disease. These and other diseases associated with M. pneumoniae are known and are contemplated to be treated by the methods of the present invention.

Antigens and Immunizing Compositions

The antigens of the invention can be included in an immunizing composition for stimulating antibody production in a subject against the antigens. The antigens can be used in clinical and research settings in known techniques and methodologies. Preferred antigens include one or more epitopes involved in binding a microorganism to a surface of a host cell. Such epitopes can be readily identified using known techniques.

The antigens may or may not be purified from the surface of the microorganism. For an immunizing composition including an isolated epitope not purified from the surface of a microorganism, the microorganism can be inactivated using known methods including, for example, heat, ether, formalin, β-propyl lactone, or attenuated by ultra-violet light, serial passaging, etc. to render the microorganism non-pathogenic. The inactivated or attenuated microorganism can then be combined with a suitable physiological carrier, for example, physiological saline, ringers solution, lactated ringers phosphate buffered saline, etc. to form a composition for administration to an animal. Immune stimulants or adjuvants can also be added to the composition to enhance the immune response. Suitable adjuvants are known and include, for example, emulsifiers, Quil A, mineral oil, aluminum hydroxide, aluminum phosphate, etc.

An immunizing composition useful for preparing antibodies can include immunologically effective amounts of both an antigen and an immunopotentiator suitable for use in mammals.

An immunopotentiator is a molecular entity that stimulates maturation, differentiation and function of B and/or T lymphocytes. Immunopotentiators are known and include T cell stimulating polypeptides such as those described in U.S. Pat. No. 4,426,324 and the C8-substituted guanine nucleosides described by Goodman et al., J. Immunol., 135:3284-88, 1985 and U.S. Pat. No. 4,643,992.

An immunizing composition is a composition containing, for example, one or more antigens of one or more translocated molecules of a microorganism or fragments thereof as an active ingredient used for the preparation of antibodies of this invention.

When a small molecule such as a polypeptide is used in an immunizing composition to induce antibodies it is to be understood that the polypeptide can be used alone or linked to a carrier as a conjugate, or as a polypeptide polymer, etc.

For a polypeptide that contains fewer than about 35 amino acid residues, it is preferable to use the peptide bound to a carrier for the purpose of inducing the production of antibodies. One or more additional amino acid residues can be added to the amino- or carboxy-termini of the polypeptide to assist in binding the polypeptide to a carrier. Cysteine residues added at the amino- or carboxy-termini of the polypeptide can be particularly useful for forming conjugates via disulfide bonds. However, other methods well known in the art for preparing conjugates can also be used.

The techniques of polypeptide conjugation or coupling through known activated functional groups have been described. See, for example, Aurameas, et al., Scand. J. Immunol., 8(Suppl. 7):7-23, 1978 and U.S. Pat. No. 4,493,795, U.S. Pat. No. 3,791,932 and U.S. Pat. No. 3,839,153. A site directed coupling reaction can also be carried out so that any loss of activity due to polypeptide orientation after coupling can be minimized. See, for example, Rodwell et al., Biotech., 3:889-894, 1985, and U.S. Pat. No. 4,671,958. Additional linking procedures including the use of Michael addition reaction products, di-aldehydes such as glutaraldehyde, Klipstein, et al., J. Infect. Dis., 147:318-326, 1983 and the like, or the use of carbodiimide technology as in the use of a water-soluble carbodiimide to form amide links to the carrier can be used. The heterobifunctional cross-linker SPDP (N-succinimidyl-3-(2-pyridyldithio) proprionate)) can also be used to conjugate peptides, in which a carboxy-terminal cysteine has been introduced.

Useful carriers are known, and generally include proteins. Carriers can include keyhole limpet hemocyanin (KLH), edestin, thyroglobulin, albumins such as bovine serum albumin (BSA) or human serum albumin (HSA), red blood cells such as sheep erythrocytes (SRBC), tetanus toxoid, cholera toxoid as well as polyamino acids such as poly (D-lysine: D-glutamic acid), and the like.

The present immunizing composition contains an effective, immunogenic amount of an antigen of a translocated molecule or fragment thereof, typically as a conjugate linked to a carrier. The effective amount of antigen of translocated molecule or fragment thereof per unit dose sufficient to induce an immune response to the immunogen depends, among other things, on the species of animal inoculated, the body weight of the animal and the chosen inoculation regimen as is well known in the art. Immunizing compositions typically contain antigen concentrations of about 10 micrograms to about 500 milligrams per inoculation (dose), preferably about 50 micrograms to about 50 milligrams per dose.

The term “unit dose” as it pertains to the immunizing composition refers to physically discrete units suitable as unitary dosages for animals, each unit containing a predetermined quantity of active material calculated to produce the desired immunogenic effect in association with the required diluent; i.e., carrier, or vehicle. The specifications for the novel unit dose of an immunizing composition are dictated by and are directly dependent on (a) the unique characteristics of the antigen and the particular immunologic effect to be achieved, and (b) the limitations inherent in the art of compounding such antigen for immunologic use in animals, as disclosed in detail herein, are features of the present invention.

Immunizing compositions can be prepared from a dried solid antigen of a translocated molecule-conjugate by dispersing the conjugate in a physiologically tolerable (acceptable) diluent such as water, saline or phosphate-buffered saline to form an aqueous composition.

Immunizing compositions can also include an adjuvant as part of the diluent. Adjuvants such as complete Freund's adjuvant (CFA), incomplete Freund's adjuvant (IFA) and alum can be used. These and other adjuvants are materials well known in the art, and are available commercially from several sources.

Antibodies

In one embodiment, the invention includes an antibody that immunoreacts with an epitope of a translocated moleule of a microorganism. Preferred antibodies immunoreact with epitopes involved in binding the surface of a host cell. The antibodies of the invention can be used in clinical and research settings in techniques and methodologies known to those of skill in the art. For example, the antibodies can be used in therapeutic, diagnostic or in vitro methods. Antibody reactivity with a stated antigen can be measured by a variety of immunological assays known in the art. Exemplary immunoreaction assays are described herein and include, for example, ELISA, Western blot, and immunoprecipitation.

Methods for preparing antibodies are known. See, Staudt et al., J. Exp. Med., 157:687-704, 1983; Examples 2 and 3 of the specification; or Antibodies: A Laboratory Manual, Harlowe and Lane, Eds., Cold Spring Harbor, N.Y. (1988). Briefly, to produce an antibody, a laboratory mammal is inoculated with an immunologically effective amount of an immunogen including an antigen of a translocated molecule, typically as present in an immunizing composition of the present invention, thereby inducing in the mammal antibody molecules having immunospecificity for the immunogen. The antibody molecules induced are then collected from the mammal and are isolated to the extent desired by well known techniques such as, for example, by immunoaffinity chromatography, or by using DEAE Sephadex™ to obtain the IgG fraction.

To enhance the specificity of the antibody, the antibody molecules can be purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. The bound antibodies can be separated from the complex by standard techniques.

Antibodies to one or more translocated molecules or fragments thereof can be used, for example, in the therapeutic and diagnostic methods and systems. For example, antibodies of the invention can be used to treat a disease in a subject due to an infection with a microorganism. The antibodies can also be used to determine whether a subject is infected with a particular microorganism. The antibodies can also be used to monitor the progression of infection and treatment of a subject.

An antibody of this invention can be a monoclonal antibody. A monoclonal antibody typically displays a single binding affinity for any epitope with which it immunoreacts. A monoclonal antibody, however, can be immunospecific for more than one epitope, e.g., a bispecific monoclonal antibody.

A monoclonal antibody is typically produced by clones of a hybridoma that produces and secretes only one kind of antibody molecule. Fusing an antibody-producing cell and a myeloma or other self-perpetuating cell line produces a hybridoma cell. Exemplary hybridoma technology is described by Niman et al., Proc. Natl. Acad. Sci., U.S.A., 80: 4949-4953, 1983. Other methods of producing a monoclonal antibody, a hybridoma cell, or a hybridoma cell culture are also well known. See, for example, Antibodies: A Laboratory Manual, Harlow et al., Cold Spring Harbor Laboratory, 1988; or the method of isolating monoclonal antibodies from an immunological repertoire as described by Sastry, et al., Proc. Natl. Acad. Sci. USA, 86: 5728-5732, 1989; and Huse et al., Science, 246: 1275-1281, 1981.

Briefly, to form a hybridoma from which the monoclonal antibody composition is produced, a myeloma or other self-perpetuating cell line is fused with lymphocytes obtained from the spleen of a mammal hyperimmunized with an immunogen. The myeloma cell line used to prepare a hybridoma can be from the same species as the lymphocytes. Mouse strains can be used. Suitable mouse myelomas for use in the present invention include the hypoxanthine-aminopterin-thymidine-sensitive (HAT) cell lines P3×63-Ag8.653, and Sp2/0-Ag14. These myelomas are available from the American Type Culture Collection, Rockville, Md., under the designations CRL 1580 and CRL 1581, respectively. Other suitable myelomas are also available from public and commercial sources. Splenocytes can be fused with myeloma cells using polyethylene glycol (PEG) 1500. Fused hybrids can then be selected by their sensitivity, for example, to HAT.

Monoclonal antibodies can also be produced by initiating a monoclonal hybridoma culture including a nutrient medium containing a hybridoma that produces and secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

A hybridoma produces a supernate that can be screened for the presence of antibody molecules that immunoreact with a translocated molecule or fragment thereof, or for inhibition of binding of a microorganism to the surface of a host cell as described further herein.

Media useful for the preparation of these compositions are both well known in the art and commercially available and include synthetic culture media, inbred mice and the like. An exemplary synthetic medium is Dulbecco's minimal essential medium (DMEM; Dulbecco et al., Virol. 8: 396, 1959)) supplemented with 4.5 gm/l glucose, 20 mm glutamine, and 20% fetal calf serum. An exemplary inbred mouse strain is the BALB/c.

Administration

The immunizing compositions of the invention may be administered by any conventional methods including oral administration and parenteral (e.g., subcutaneous or intramuscular) injection. The treatment may consist of a single dose of immunizing composition or a plurality of doses over a period of time. The immunogen can include one or more epitopes of one or more translocated molecules of a microorganism. Suitable methods of administration are disclosed in, for example, PCT Patent publucation WO 00/42068.

The immunizing composition can include an adjuvant. The proportion of immunogen and adjuvant can be varied over a broad range so long as both are present in effective amounts. For example, aluminum hydroxide can be present in an amount of about 0.5% of the mixture (Al₂O₃ basis). On a per-dose basis, the amount of the immunogen can range from about 5 μg to about 100 μg protein per patient of about 70 kg. A range from about 20 μg to about 40 μg per dose is preferred. A suitable dose size is about 0.5 ml. Accordingly, a dose for intramuscular injection, for example, would include 0.5 ml containing 20 μg of immunogen in admixture with 0.5% aluminum hydroxide.

The therapeutic application of immunizing compositions can be done by way of nasal administration. Various ways of such administration are known in the art. The pharmaceutical formulation for nasal administration may be prepared as solutions in saline, employing benzyl alcohol or other suitable preservatives, absorption promoters to enhance bioavailability, fluorocarbons, and/or other solubilizing or dispersing agents known in the art. The unit dosage for nasal administration can be from 1 to 3000 mg, preferably 70 to 1000 mg, and most preferably, 1 to 10 mg of active ingredient per unit dosage form.

Other modes of administration including suppositories and oral formulations can be used and may be desirable.

Antigens can also be administered in conjunction with immune stimulating complexes. Immune stimulating complexes are negatively charged cage-like structure of 30-40 nm in size formed spontaneously on mixing cholesterol and Quil A (saponin). Protective immunity has been generated in a variety of experimental models of infection including toxoplasmosis and Epstein-Barr virus-induced tumors using immune stimulating complexes as the delivery vehicle for antigens (see, e.g., Mowat and Donachie, Immunol. Today, 23: 383-385, 1991). Immunizing compositions using immune stimulating complexes include antigens encapsulated into immune stimulating complexes for delivery.

Immunotherapy regimens which produce maximal immune responses following the administration of the fewest number of doses, ideally only one dose, are highly desirable. This result can be approached through entrapment of immunogen in microparticles. For example, the absorbable suture material poly(lactide-co-glycolide) co-polymer can be fashioned into microparticles containing immunogen (see, e.g., Eldridge et al., Molec. Immunol., 28: 287-294, 1991; Moore et al., Vaccine 13: 1741-1749, 1995; and Men et al., Vaccine, 13: 683-689, 1995). Following oral or parenteral administration, microparticle hydrolysis in vivo produces the non-toxic byproducts, lactic and glycolic acids, and releases immunogen largely unaltered by the entrapment process.

Microparticle formulations can also provide primary and subsequent booster immunizations in a single administration by mixing immunogen entrapped microparticles with different release rates. Single dose formulations capable of releasing antigen ranging from less than one week to greater than six months can be readily achieved.

Passive Immunization

In one embodiment of the invention, passive immunization is used to treat a disease in a subject due to an infection with a microorganism. Passive immunization means administration of antibodies to a subject. Passive immunization can be accomplished with polyclonal antibodies, monoclonal antibodies, or antibody fragments. In one embodiment, passive immunization methods include administering a composition including more than one species of monoclonal antibody. Preferably, the antibodies are directed to epitopes involved in binding a microorganism to the surface of a host cell.

Generally, the dosage will vary with the age, condition, sex and extent of the disease in the subject and can be determined by one of skill in the art. A therapeutically effective amount of an antibody of this invention is typically an amount of antibody such that when administered in a physiologically tolerable composition is sufficient to achieve a plasma concentration of from about 0.1 μg/ml to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

Antibodies can be administered parenterally by injection or by gradual infusion over time. Antibodies can be administered intravenously, intraperitoneally, intramuscularly, subcutaneously, intracavity, transdermally, and can be delivered by peristaltic means.

Compounds, Compositions and Methods Thereof

In another embodiment, the invention provides compounds for inhibiting binding of a microorganism to the surface of a host cell. The compounds of the invention are useful in methods for inhibiting binding of a microorganism to the surface of a host cell or a molecule thereof. Binding of a microorganism to the surface of a host cell or a molecule thereof is inhibited by contacting a microorganism, with an effective inhibitory amount of a compound of the invention. Compounds of the invention can include small organic molecules, either naturally produced, for example by a microorganism or plant, or synthetically produced; and macromolecules; including lipids, polypeptides, carbohydrates, and nucleic acids, or combinations thereof.

Examples of compounds useful for inhibiting binding of a microorganism to a surface of a host cell include mucin-associated sugars. Mucin associated sugars include fucose, N acetylgalactosamine, N-acetylglucosamine, sialic acid, and galactose.

In one embodiment, the compound is the translocated molecule or a protein thereof. The compound can be a species homolog of a translocated molecule.

In one embodiment, the invention provides compounds that interact with a translocated molecule of a microorganism and interfere with the binding of the translocated molecule with the surface of a host cell or a molecule thereof. Interaction of a translocated molecule with a compound of the invention can include hydrogen bonding, van der Waal's interactions, ionic interaction, covalent binding, and the like.

In another embodiment, the invention provides compounds that when contacted with a microorganism result in the internalization of a translocated molecule of the microorganism. As used herein, “internalization” means actively or passively transporting a molecule located on the surface of a cell into the cytosol. Mechanisms of internalization are known. For example, internalization can include endocytosis. Typically, internalization is an active process

In yet another embodiment, the invention provides compounds that directly or indirectly degrade a translocated molecule. As used herein, “degrade” or “degradation” means to remove one or more atoms from a molecule. Degradation typically involves the cleavage of bonds within a molecule. Compounds that degrade translocated molecules can include polypeptide enzymes and catalytic RNA molecules.

Screening of compounds for ability to inhibit binding of either isolated translocated molecules or microorganisms to the surface of a host cell or molecules thereof can be performed using methods described herein and/or other known techniques. It will be appreciated that inhibition can occur through a variety of mechanisms.

In another embodiment, the invention provides methods for inhibiting the binding of a microorganism or a translocated molecule thereof to the surface of a host cell or a molecule thereof. The method comprises contacting a microorganism or a translocated molecule thereof with one or more compounds; wherein the one or more compounds (a) interact with a translocated molecule of a microorganism and interfere with the binding of the translocated molecule with the surface of a host cell or a molecule thereof, (b) compounds directly or indirectly cause the internalization of a translocated molecule of the microorganism; (c) directly or indirectly degrade a translocated molecule; or (d) a combination thereof.

In another embodiment the invention provides a method for treating a disease in a subject due to infection with a microorganism. The method comprises administering to the subject an effective amount of one or more compounds or pharmaceutically acceptable salts thereof, wherein the one or more compounds or pharmaceutically acceptable salts thereof (a) interact with a translocated molecule of a microorganism and interfere with the binding of the translocated molecule with the surface of a host cell or a molecule thereof, (b) compounds directly or indirectly cause the internalization of a translocated molecule of the microorganism; (c) directly or indirectly degrade a translocated molecule; or (d) a combination thereof.

The invention also provides compositions comprising one or more compound of the invention. The compositions can be pharmaceutical compositions.

A compound or inhibitor of the invention is preferably administered in combination with a pharmaceutically acceptable carrier, and may be combined with specific delivery agents, including targeting antibodies and/or cytokines. The compound or inhibitor of the invention may be administered in combination with other pharmaceutically active agents. A compound of the invention can be administered in combination with other agents useful in the treatment of disease due to infection with a microorganism. For example, a compound can be administered in combination with effective amounts of an antimicrobial agent, including an antibacterial agent, an antifungal agent, an antimycoplasmal agent, and an antibody or immunizing composition of the invention.

The compounds of the invention can be administered orally, parentally (including subcutaneous injection, intravenous, intramuscular, intrasternal or infusion techniques), by inhalation spray, topically, by absorption through a mucous membrane, or rectally, in dosage unit formulations containing conventional non-toxic pharmaceutically acceptable carriers, adjuvants or vehicles. Pharmaceutical compositions of the invention can be in the form of suspensions or tablets suitable for oral administration, nasal sprays, creams, sterile injectable preparations, such as sterile injectable aqueous or oleagenous suspensions or suppositories.

For oral administration as a suspension, the compositions can be prepared according to known pharmaceutical formulation techniques. The compositions can contain microcrystalline cellulose for imparting bulk, alginic acid or sodium alginate as a suspending agent, methylcellulose as a viscosity enhancer, and sweeteners or flavoring agents. As immediate release tablets, the compositions can contain microcrystalline cellulose, starch, magnesium stearate and lactose or other excipients, binders, extenders, disintegrants, diluents and lubricants known in the art.

For administration by inhalation or aerosol, the compositions can be prepared according to techniques well-known in the art of pharmaceutical formulation. The compositions can be prepared as solutions in saline, using benzyl alcohol or other suitable preservatives, absorption promoters to enhance bioavailability, fluorocarbons or other solubilizing or dispersing agents known in the art.

For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

For rectal administration as suppositories, the compositions can be prepared by mixing with a suitable non-irritating excipient, such as cocoa butter, synthetic glyceride esters or polyethylene glycols, which are solid at ambient temperatures, but liquify or dissolve in the rectal cavity to release the drug.

Dosage levels of approximately 0.02 to approximately 10.0 grams of a compound of the invention per day are useful in the treatment of a disease due to an infection with a microorganism, with oral doses 2 to 5 times higher. For example, disease due to infection with a microorganism can be treated by administration of from about 0.1 to about 100 milligrams of compound per kilogram of body weight from one to four times per day. In one embodiment, dosages of about 100 to about 400 milligrams of compound are administered orally every six hours to a subject. The specific dosage level and frequency for any particular subject will be varied and will depend upon a variety of factors, including the activity of the specific compound, the metabolic stability and length of action of that compound, the age, body weight, general health, sex, and diet of the subject, mode of administration, rate of excretion, drug combination, and severity of the particular condition.

A compound of the invention can be administered in combination with other agents useful in the treatment disease due to infection with a microorganism. For example, a compound can be administered in combination with effective amounts of an antimicrobial agent, including an antibacterial agent, an antifungal agent, an antimycoplasmal agent, and an antibody or immunizing composition of the invention. The compound of the invention can be administered prior to, during, or after a period of actual or potential exposure to microorganism.

The following examples are offered for illustrative purposes only, and are not intended to limit the scope of the present invention in any way. All patent and literature references cited in the present specification are hereby incorporated by reference in their entirety. All parts and percentages are by weight unless otherwise specified.

### EXAMPLE 1

### Antibodies to EF-Tu and Pyruvate Dehydrogenase of Mycoplasma pneumoniae Inhibit Binding of the Mycoplasma to Fibronectin

Mycoplasma pneumoniae is considered among the smallest self-replicating procaryotic pathogens with a genome size of 800 kbp and utilizes a unique terminal tip organelle to mediate adherence to target cells. This tip-mediated adherence involves a network of mycoplasma proteins including adhesins and adherence-accessory proteins. Many of these proteins appear to be homologues of cytoskeletal-like proteins of eucaryotes and function in the mobilization and concentration of adhesins at the mycoplasma tip structure. Spontaneous hemadsorption-negative (HA⁻) mutants, which exhibit markedly reduced binding to a variety of eucaryotic cells, fail to express many of these adherence-related proteins. Spontaneous hemadsorption-positive (HA⁺) revertants to these mutants resynthesize these proteins and regain cytadherence and virulence capabilities. Furthermore, polyclonal and monoclonal Abs directed against M. pneumoniae tip-associated adhesins markedly inhibit binding of M. pneumoniae to hamster tracheal rings. However, we have considered the possible existence of alternate mechanisms of mycoplasma adherence based upon electron and confocal microscopy and HA⁻ mutant analyses that implicated non-tip mediated mechanisms of colonization and entry into host target cells.

In this example we report that two M. pneumoniae cytoplasmic proteins with well-known biosynthetic and metabolic activities, elongation factor Tu (EF-Tu) and pyruvate dehydrogenase E1 β subunit (PDH-B), exhibit a surface location and an unexpected and novel function, that is the mediation of M. pneumoniae binding to Fn. These observations provide new insights concerning the biological versatility of M. pneumoniae and implicate alternative mechanisms by which mycoplasmas parasitize host targets.

### Methods

Mycoplasma and Culture Conditions.

Wild-type HA⁺ M. pneumoniae (clinical isolate designated S1) and P30 adhesin-deficient HA⁻ class II mutant 15 (Krause et al., Infect Immun, 35: 809-817, 1982) were grown to late logarithmic phase in SP-4 medium at 37° C. for 72 h in 150-cm² tissue culture flasks (Baseman et al., J Bacteriol, 151: 1514-1522, 1982). Mycoplasmas were harvested by washing three times with PBS [150 mM NaCl, 10 mM sodium phosphate, pH 7.4] and pelleting at 12,500 g for 15 min at 4° C. (Dallo et al., Infect Immun, 64: 2595-2601, 1996). For radiolabeling, surface-attached M. pneumoniae grown in flasks were washed three times with PBS, scraped and collected by centrifugation at 12,500 g. Mycoplasma cells were biosynthetically labeled with [³⁵S]-methionine as follows: mycoplasmas were resuspended in one tenth of their original volume in DMEM without cysteine or methionine but supplemented with 10% fetal bovine serum. 1 mCi [³⁵S]-methionine was added, and cells were incubated at 37° C. on a rocker for 4 h, pelleted and washed four times with PBS.

Bacterial Strains, Plasmids and DNA Manipulations.

Escherichia coli INαF′[F′endA1rec1hsdR17supE44gyrA96lacZM15 (lacZYAargF)] (Invitrogen) and E. coli BL21(DE3) [F′ompT hsdS (r_{B}⁻ m_{B}⁻) gal dcm λ(DE3) pLysS] (Stratagene) were grown in Luria Bertani (LB) broth and used to clone and express mycoplasma EF-Tu and PDH genes. For DNA manipulations, the following vectors were used: pCR2.1 (Ap^{r}, Km^{r} TA cloning vector [Invitrogen]) and pET16b (Ap^{r}, N-terminal His¹⁰ tag, expression vector [Novagen]). M. pneumoniae DNA was prepared as described previously (Dallo et al., Infect Immun, 58: 4163-4165, 1990; Dallo et al., Infect Immun, 57: 1059-1065, 1989). Plasmid DNA was purified using the QIAprep spin protocol according to the manufacturer (Qiagen).

Identification of FnBPs by Ligand Immunoblot Assay.

Mycoplasma proteins were separated on 12% SDS-polyacrylamide gels (Laemmli, Nature, 227: 680-685, 1970) and transferred electrophoretically to nitrocellulose membranes (Towbin et al., Proc Natl Acad Sci USA, 76: 4350-4354, 1979). Membranes were blocked for 1 h at 25° C. with 3% (wt/vol) blotto [nonfat dry milk in PBS containing 0.05% Tween-20 (PBST)], followed by two washes with PBST, and incubated for 24 h at 4° C. with human Fn (20 μg/ml, Sigma) in 1% blotto. Then, individual membranes were washed three times (15 min per wash) in PBST and incubated for 2 h (ambient temperature) with rabbit anti-Fn Abs at a 1:1000 dilution in 1% blotto. Subsequently, the blots were washed three times (15 min per wash), incubated for 1 h at 25° C. with peroxidase-conjugated goat anti-rabbit Abs at a dilution of 1:3000 in 1% blotto, washed 3 additional times with PBST, then once with PBS and developed with 4-chloro-1-naphthol in PBS and H₂O₂ (0.5%).

Dose-Dependent Binding of M. pneumoniae to Immobilized Fn.

Individual wells of microtitre plates (Immunoplate I; Nunc) were coated overnight with 100 μl of 0.01-100 μg/ml solution of Fn in PBS and washed twice with PBS. Unoccupied sites were blocked with 200 μl of 1 mg/ml BSA in PBST for 1 h at 37° C. Wells coated with BSA alone served as negative controls. [³⁵S]-methionine-labeled M. pneumoniae (100 μl, 10⁷ cells/well) were added to each well, and microtitre plates were incubated at 37° C. for 2 h and washed four times with PBST to remove nonadherent mycoplasmas. Microtitre wells were detached and dissolved in scintillation fluid for radioactive determinations.

Purification of FnBPs.

Radiolabeled M. pneumoniae cells were resuspended in 20 ml of TDSET (10 mM Tris [pH 7.8], 0.2% sodium deoxycholate, 0.1% SDS, 10 mM tetrasodium EDTA, 1% Triton X-100) containing 1 mM PMSF, repeatedly forced through a 25-gauge needle to facilitate lysis, and incubated on a rocker for 30 min at room temperature. Unbroken cells and cellular debris were removed by centrifugation at 14,000 g. The supernatant was incubated with Fn-coupled Sepharose (2 mg of Fn coupled to 1 g of CNBr-activated Sepharose according to the manufacturer's directions, except for the coupling buffer which was 100 mM sodium bicarbonate, pH 8.3) on a rocker at 4° C. overnight. The affinity column was washed with coupling buffer, and radioactive fractions were eluted with 5 M LiCl, concentrated using an Amicon concentrator and tested for Fn binding activity in the ligand immunoblot assay. Parallel experiments were performed with uncoupled Sepharose to reveal non-specific binding and with unlabeled mycoplasmas to determine N-terminal protein sequencing.

N-Terminal Protein Sequencing.

PVDF membrane (Immunobilion P; Millipore Corp.) blots of SDS-polyacrylamide gels containing M. pneumoniae FnBPs were stained with 0.1% Ponceau S solution (wt/vol) and washed thoroughly in distilled water. Individual protein bands were excised from the blot and subjected to Edman degradation sequencing by the microsequencing facility at Baylor College of Medicine (Houston, Tex.).

Cloning and Sequencing of EF-Tu and PDH-B.

Based on the published sequence of the M. pneumoniae genome (Himmelreich et al., Nucleic Acids Res, 24: 4420-4449, 1996), the complete open reading frame of EF-Tu was amplified using the forward primer 5′-GAGACGTAATTCAAACATATGGCAAGAG AG-3′ (SEQ ID NO: 3) and the reverse primer 5′-GGCTTTCCTTGAGGATCCT AACAGAGTCAA-3′ (SEQ ID NO: 4), which produces NdeI and BamHI (underlined) sites at the 5′ and 3′ ends of the EF-Tu ORF, respectively. For PDH-B approximately three-fourths of the open reading frame of the pdh gene (825-bp DNA fragment that encodes truncated peptide of 244 aa from the NH₂-terminal and designated NPDH due to UGA encoding tryptophan at aa 245) was amplified by PCR using the forward primer 5′-ATTAATAAATTCCATATGTCAAAAACAATTCAA-3′ (SEQ ID NO: 5) and the reverse primer 5′- AGCCGCTTCGGTAACCTCGAGCAAGCG-3′ (SEQ ID NO: 6), which produces NdeI and XhoI (underlined) sites at the 5′ and 3′ ends of the pdh ORF, respectively. Both fragments were ligated into the pCR 2.1 vector and transformed into E. coli INVαF′ cells for automated sequencing using M13 forward and reverse primers. Subsequent primers were designed based on the sequences obtained.

Expression and Purification of Recombinant Proteins.

DNA fragments generated by digesting plasmid pCR-EF-Tu with NdeI and BamHI and plasmid pCR-NPDH with NdeI and XhoI were ligated into pET16b to generate pET-EF-Tu and pET-NPDH, respectively. These plasmids were transformed into competent E. coli BL21 (DE3) cells that were grown to a density of 2×10⁹ cells/ml at 37° C. in standard LB broth containing 100 μg/ml ampicillin (Sigma-Aldrich). Induction of recombinant protein synthesis was accomplished by the addition of 100 μM of isopropyl thio β-galactopyranoside (Sigma-Aldrich), and bacteria were incubated for 3 h at 37° C. under aeration at 220 rpm. Cells from 1 ml samples were pelleted, resuspended in 250 μl of sample buffer (4% SDS, 125 mM Tris (pH 6.8), 10% 2-ME, 10% glycerol, 0.2% bromophenol blue), and heated to 95° C. for 5 min. 10 μl aliquots of test samples were analyzed on 12% SDS/polyacrylamide gels. Recombinant colonies were screened for resistance to ampicillin and expression of a protein product of the correct size, and one recombinant clone from each construct was selected for further study. Verification of specific clones was achieved by restriction digestion and limited DNA sequencing. Fusion proteins were purified from urea lysates of recombinant E. coli by nickel affinity chromatography using the manufacturer's denaturing protocol (Qiagen).

Preparation of Antisera Against Recombinant Mycoplasma Proteins.

New Zealand White rabbits were immunized subcutaneously with 100-200 μg of recombinant proteins suspended in complete Freund's adjuvant. Individual rabbits were boosted three times with the same amount of antigen in IFA every 21 days. Serum samples were collected and used for immunological characterization and competition binding assays.

Whole-Cell Radioimmunoprecipitation (WCRIP).

To determine Ab-accessible epitopes of EF-Tu and PDH-B on the surface of M. pneumoniae, WCRIP with intact, viable [³⁵S]-methionine-labeled mycoplasmas was performed. Radiolabeled mycoplasmas in PBS were divided into aliquots to which were added anti-rEF-Tu and anti-rNPDH antisera, or preimmune sera as negative controls. Mycoplasma suspensions were placed on a rocker platform for 90 min at 4° C., and mycoplasmas were pelleted and washed 2 times with PBS to remove unabsorbed Abs. Cell pellets were resuspended in 1 ml TDSET/PMSF (10 mM Tris-HCl [pH 7.8], 0.2%[wt/vol] sodium deoxycholate, 0.1%[wt/vol] SDS, 10 mM EDTA, and 1%[vol/vol] Triton X-100 that contained 100 mM PMSF), vortexed and incubated at 37° C. for 60 min with periodic vortexing to ensure efficient solubilization. Insoluble material was removed by centrifugation at 45,000 g for 60 min. 0.9 ml of each supernatant was carefully transferred to another tube, and 250 μl of washed Staph A were added to each supernatant. Test suspensions were placed on a rocker platform for 90 min at 4° C., and Staph A with adsorbed immune complexes were pelleted and washed four times with TDSET. Adsorbed M. pneumoniae surface immunogens were eluted by resuspending Staph A pellets in 35 μl SP buffer (0.1M Tris-HCl [pH 6.8], 2%[wt/vol] SDS, 20%[vol/vol] glycerol, 2%[wt/vol] 2-ME, and 0.02%[wt/vol] bromophenol blue) and boiling the suspension for 3 min. Then, Staph A were pelleted, and supernatants were subjected to SDS-PAGE. WCRIP assays were also performed using [³H]-thymidine-labeled mycoplasmas to assess the extent of mycoplasma cell lysis during the initial Ab incubation, which was less than 3%.

To examine trypsin sensitivity of EF-Tu and PDH-B proteins, trypsin type III (bovine pancreas—Sigma) was used. Briefly, intact mycoplasma cells (400 μg of total protein) in 1 ml of PBS were added to microfuge tubes, and each sample was incubated with 10 and 50 □g of trypsin for 30 min at 37° C. A cocktail of protease inhibitors (Sigma) was added, and incubation was continued at 0° C. for 10 min. Samples were then centrifuged, analyzed by WCRIP, subjected to SDS-PAGE and transferred to nitrocellulose for immunoblotting with anti-rEF-Tu and anti-rNPDH-BAbs.

Immunogold Electron Microscopy

Fresh intact M. pneumoniae cells were washed in 100 mM Tris-HCl buffer (pH 7.5) and incubated with 100 mM Tris-HCl buffer (pH 7.5) containing 1% bovine serum albumin (BSA) supplemented with 1% heat inactivated goat serum (buffer A) to reduce nonspecific binding. For single gold particle labeling, cells were incubated 120 min at 37° C. with anti-rEF-Tu or anti-rNPDH-B sera diluted (1:100) in buffer A. Mycoplasmas were then washed with buffer A and incubated for 60 min at room temperature with goat anti-rabbit immunoglobulin G (IgG)-gold complex (average size particle, 10 nm, 1:20 dilution) suspended in PBS (pH 7.4) containing 1% BSA (buffer B). After sequential washing with filtered (0.22 □m, Millipore) buffer B, PBS, and deionized water, mycoplasmas were mounted onto Formvar-coated nickel grids by fixing with 1% glutaraldehyde-4% formaldehyde for 20 min at room temperature. For double gold particle labeling, cells were washed and blocked with Buffer A as above, and incubated sequentially with anti-PDH-B sera followed by 10 nm IgG gold particles. Then, individual grids were gently washed, fixed, washed again and blocked with Buffer A, followed by anti-EF-Tu sera and IgG gold particles (20 nm). Additional washing and fixation steps followed. Finally, grids were stained with 7% uranyl acetate followed by Renolyds lead citrate and examined with a Philips 208S Transmission Electron Microscope at ˜60 kv accelerating voltage.

Competition Binding Assays.

Rabbit sera raised against rEF-Tu and rNPDH were used to block M. pneumoniae binding to Fn on microtitre plates. Anti-rEF-Tu and anti-rNPDH sera at final dilutions of 1:100, 1:250 and 1:1000 were added individually or together to biosynthetically radiolabeled mycoplasmas prior to the assessment of M. pneumoniae binding to Fn. Pre-immune sera at the same dilutions were used as negative controls.

Computer Assisted Analysis.

Amino acid identity matches were performed using the National Center for Biotechnology Information's sequence similarity search tool designed to support analysis of nucleotide and protein databases at http://www.ncbi.nlm.nih.gov/BLAST. All M. pneumoniae sequence data used in this study were downloaded from the database at http://www.zmbh.uniheidelberg.de/M_{—}pneumoniae/MP_Home.html

Results

Fn Binding of M. pneumoniae Proteins.

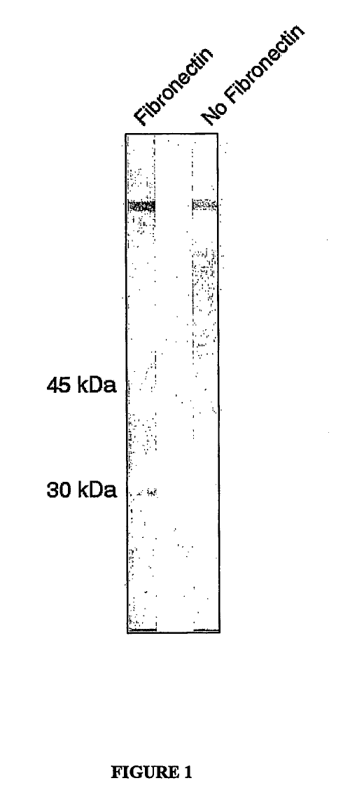

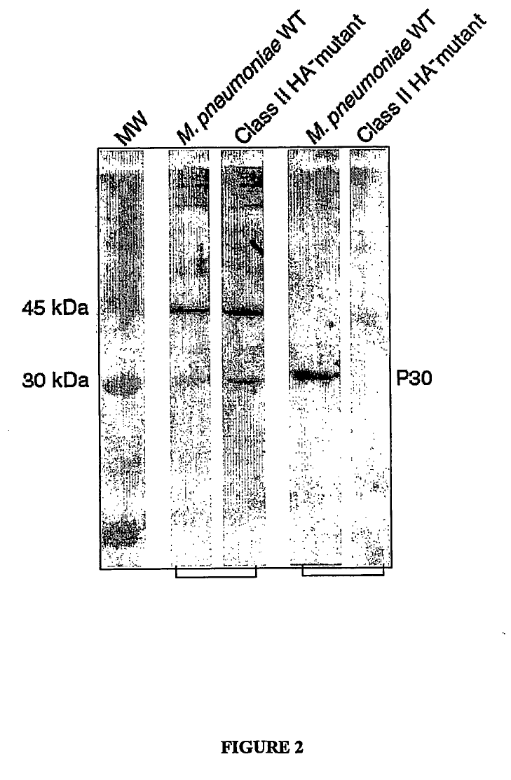

Our initial attempts to identify FnBPs of M. pneumoniae utilized a ligand immunoblot assay that assessed the ability of M. pneumoniae proteins separated by SDS-PAGE to bind human plasma Fn. As shown in FIG. 1, two distinct mycoplasma proteins (45-kDa and 30-kDa) bound specifically to Fn. The 30-kDa protein closely migrates with the well-characterized P30 adhesin of M. pneumoniae, which is known to exhibit immunological cross-reactivity with mammalian structural proteins, like human keratin, myosin and fibrinogen (Baseman et al., Am J Respir Crit Care Med, 154: S137-144,1996). Therefore, we probed total SDS-PAGE protein extracts of wild-type M. pneumoniae and class II HA⁻ mutants [previously isolated and characterized by us as lacking the P30 adhesin (Baseman et al., Isr J Med Sci, 23: 474-479, 1987)] with both anti-P30 adhesin Abs as well as Fn plus anti-Fn Abs. Positive Fn-binding signals were observed in both wild-type and mutant strains (FIG. 2), confirming the uniqueness of the 30-kDa FnBP.

Fn-Affinity Column Chromatography of M. pneumoniae Proteins.

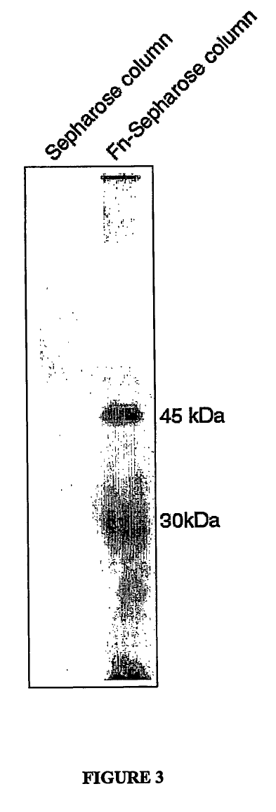

To further assess the specificity of M. pneumoniae FnBPs, we applied total mycoplasma [³⁵S]-methionine-labeled protein lysates to human Fn-coupled affinity columns. Mycoplasma proteins, which specifically bound to Fn, were eluted with 5 M LiCl. Fractions containing high radioactive counts were concentrated, subjected to SDS-PAGE and transferred to nitrocellulose membranes. As shown in FIG. 3, a distinct protein of 45-kDa and a broader protein band at 30 to 35-kDa were detected. The sizes of these two proteins correspond directly with the mycoplasma proteins bound in the Fn ligand immunoblot assay (FIG. 1). Although not detectable by Coomassie brilliant blue staining, several minor bands between 14 to 18-kDa were observed in the column eluent based upon [³⁵S]-methionine labeling or silver staining, but none bound Fn.

Cloning and Expression of FnBPs.

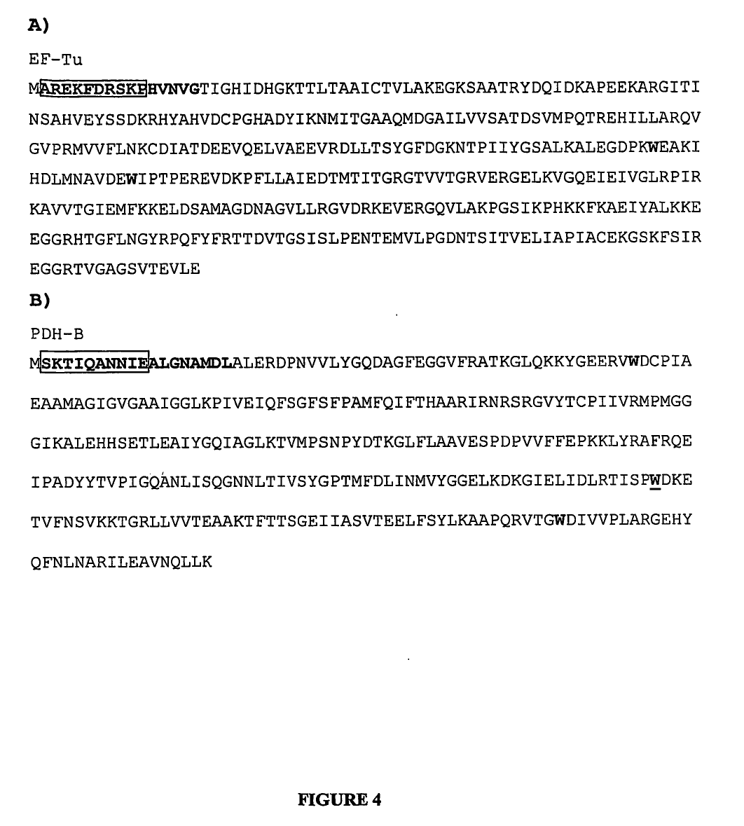

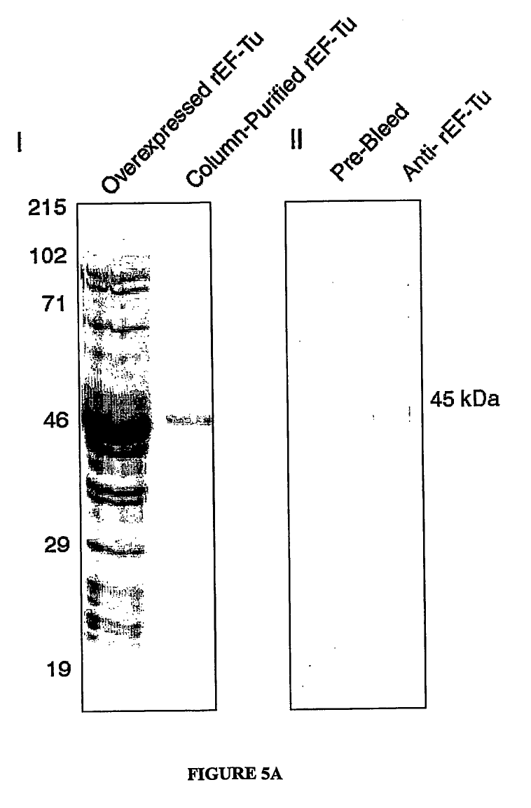

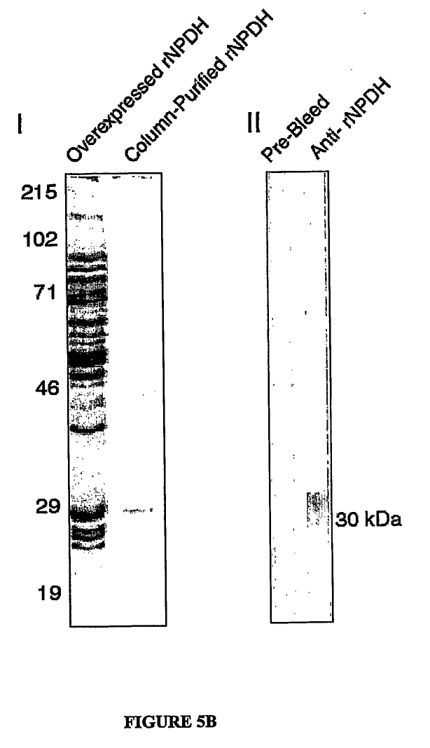

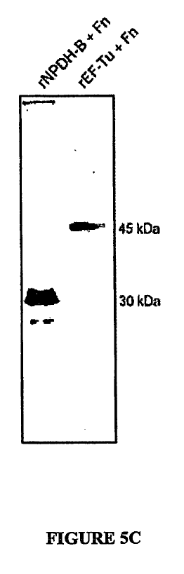

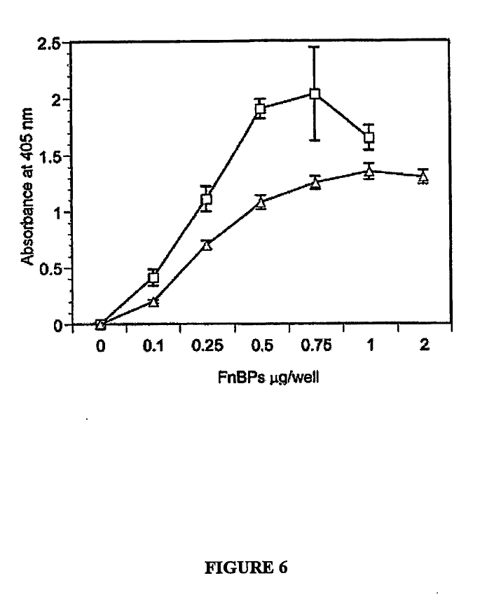

To assure that the FnBPs identified by the ligand immunoblot assay and Fn column chromatography were the same, we subjected parallel protein samples of the 45- and 30-kDa proteins to NH₂-terminal amino acid sequencing. All sequences exhibited perfect matches and revealed 100% identity with the NH₂-terminal region of EF-Tu (45-kDa) of M. pneumoniae (FIG. 4A) and PDH-B (30-kDa) of M. pneumoniae (FIG. 4B). However, to obtain sufficient amounts of M. pneumoniae EF-Tu and PDH-B for additional binding assays and for the generation of antisera, we utilized the His-tag expression system and Ni(II)-NTA resin chromatography to express and purify recombinant mycoplasma proteins. Since mycoplasmas use both UGA (“universal” stop codon) and UGG to encode tryptophan, we analyzed the nucleotide and amino acid sequences of EF-Tu and PDH-B for UGA-encoded tryptophan. No UGA codons appeared in the EF-Tu gene sequence, which predicted a protein of 394 amino acids (FIG. 4A). In contrast, PDH-B featured a single UGA-encoded tryptophan at amino acid position 245 (FIG. 4B). Thus, the gene encoding PDH-B, which is predicted to encode a protein of 327 amino acids (FIG. 4B) would be truncated in E. coli. As appears in FIG. 5A-I the complete EF-Tu gene was cloned and expressed as a His¹⁰-tagged protein (His¹⁰-EF-Tu; Theoretical Mw of 45668.97 Da), whereas the E. coli PDH-B expressed gene encoded a truncated 244 amino acid recombinant protein fused at the NH₂-terminal with the His-tag and designated NPDH-B (His¹⁰-NPDH-B; Theoretical Mw of 29129.42 Da) (FIG. 5B.I). Both rEF-Tu and rNPDH were purified to homogeneity, and rabbit polyclonal Abs were raised against these proteins (FIG. 5A. II and B. II). Anti-rEF-Tu antiserum recognized a single band by immunoblot blot around 43-kDa (FIG. 5AII) whereas anti-rNPDH antiserum recognized a broader band around 30 to 35-kDa (FIG. 5BII). Ligand immunoblot analysis of the E. coli expressed recombinant mycoplasma proteins revealed Fn binding activities associated with the 45- and 30-kDa recombinant proteins (FIG. 5C). The Fn binding activity of the recombinant EF-Tu and PDH-B proteins was also assessed by ELISA (FIG. 6). Bothe rEF-Tu and rNPDH-B bound to immobilized Fn in a dose dependent, saturable manner. The dissociation constant (kd) value for binding of EF-Tu with Fn was 50 ng/ml and pf PDH-B was 75 ng/ml, and these determinations fall well within expected physiological values.

Surface Location of EF-Tu and PDH-B.

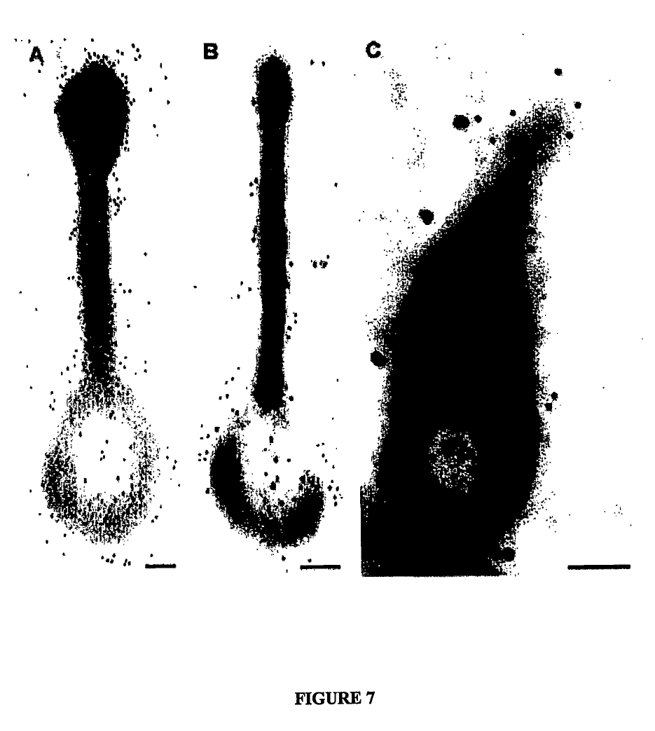

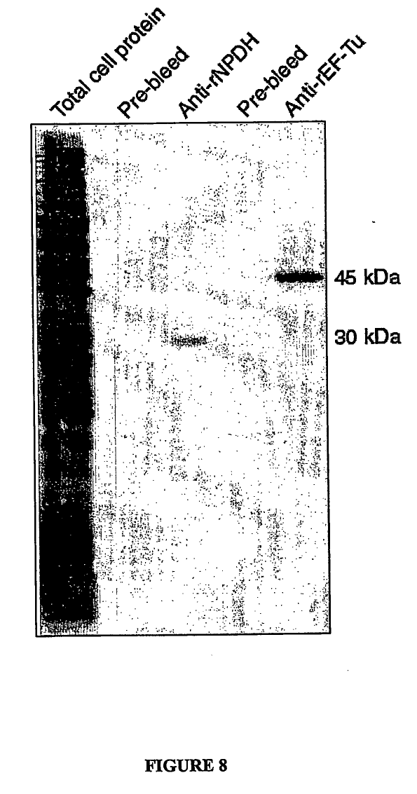

Both EF-Tu and PDH-B are considered cytoplasmic proteins. EF-Tu is an essential component in protein synthesis, and PDH-B is involved in pyruvate oxidation. The unexpected Fn binding properties of these proteins led us to examine their possible surface location in M. pneumoniae. Polyclonal antibodies which specifically recognize recombinant FnBPs were used to examine intact mycoplasmas by immunogold electron microscopy. Immunogold labeling with anti-rNPDH-B (FIG. 7A, 10 nm gold particles) or anti-rEF-Tu (FIG. 7B, 10 nm gold particles) Abs revealed membrane and tip-concentrated labeling (PDH-B) and random membrane labeling (EF-Tu) of gold particles on M. pneumoniae cell surfaces. This was further reinforced by double labeling experiments in which mycoplasmas were first preincubated with anti-rNPDH-B sera and 10 nm IgG gold-conjugated particles, followed by anti-recombinant NPDH-B sera ans 10 nm IgG gold-conjugated particles, followed by anti-recombinant EF-Tu sera and 20 nm IgG gold conjugated particles (FIG. 7C). Prebleed control sera, which were subsequently exposed to gold-conjugated Abs, were free of label. To further confirm the surface location of FnBPs, we performed whole-cell radioimmunoprecipitation (WCRIP) using biosynthetically [³⁵S]-methionine-labeled intact, viable M. pneumoniae incubated with anti-rEF-Tu and anti-rNPDH antisera and prebleed controls. As shown in FIG. 8, WCRIP performed with specific immune sera demonstrated the surface accessibility of EF-Tu and PDH-B epitopes, which appeared as 45 kDa and 30 kDa proteins, respectively. To identify whether surface accessible EF-Tu and PDH-B epitopes were trypsin sensitive, we performed WCRIP on trypsin-treated or untreated intact mycoplasmas. Trypsin (10 μg/ml for 30 min) reduced surface immunoreactivity of PDH-B by greater than 95%. In contrast, EF-Tu was unaffected by trypsin at this concentration. However, a trypsin concentration of 50 μg/ml for 30 min completely removed EF-Tu immunoreactivity. The tip-associated, trypsin-sensitive P1 adhesion, served as a positive control (Baseman et al., Subcell Biochem, 20:243-259, 1993).

Binding of M. pneumoniae to Immobilized Fn.

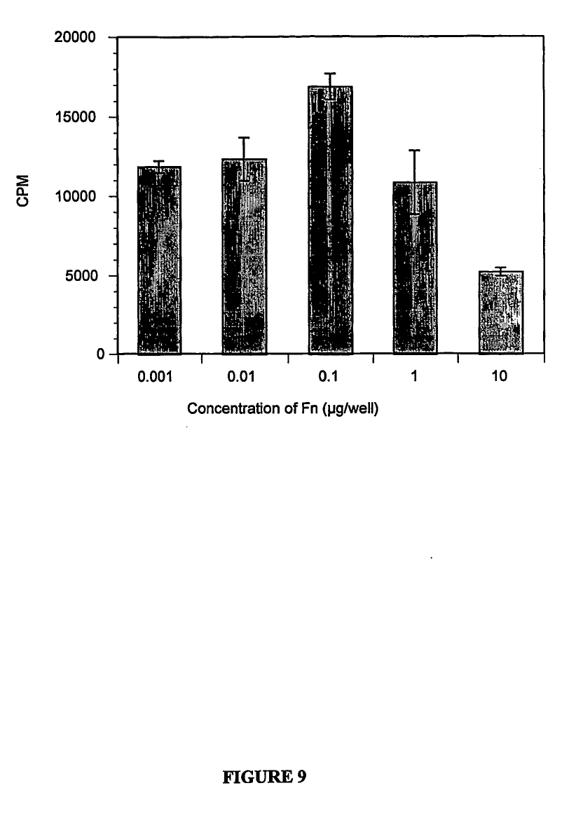

To further assess the role of FnBPs in M. pneumoniae adherence, we monitored the ability of M. pneumoniae to interact with Fn immobilized on wells of microtiter plates. Biosynthetically radiolabeled viable M. pneumoniae were added to microtitre wells coated with increasing concentrations of Fn (0.001 to 10 μg/well). Maximal mycoplasma binding was observed at a Fn concentration of 0.1 μg/well (FIG. 9) whereas further increases in Fn concentrations resulted in reduced mycoplasma binding. A similar decrease in binding to high Fn concentrations has been reported in Aspergillus fumigatus (Penalver et al., Infect Immun, 64: 1146-1153, 1996).

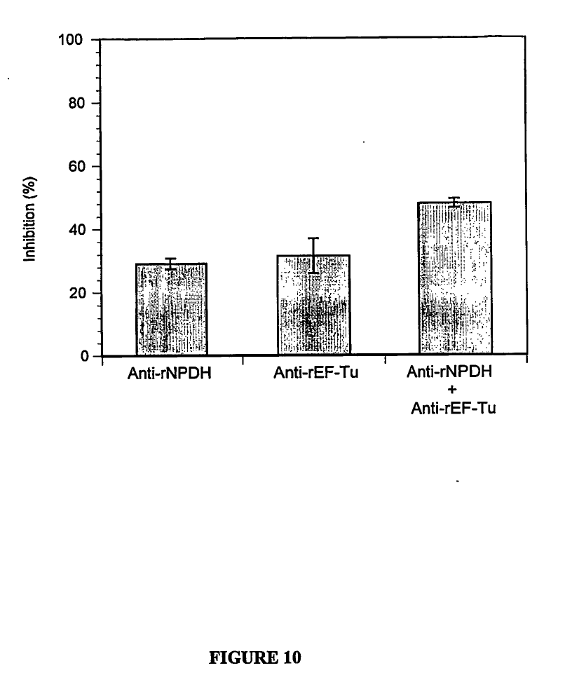

Preincubation of [³⁵S]-methionine-labeled M. pneumoniae with anti-rEF-Tu and anti-rNPDH Abs blocked mycoplasma binding to Fn. Anti-rEF-Tu at dilutions of 1:1000, 1:500 and 1:100 inhibited mycoplasma binding to 0.1 μg/ml Fn by 14, 24 and 31%, respectively. Similarly, the same dilutions of anti-rNPDH antisera inhibited mycoplasma binding to Fn by 17, 23, and 30%, respectively. As shown in FIG. 10, pre-incubation of M. pneumoniae with both anti-EF-Tu and anti-NPDH Abs at 1:100 dilutions acted cumulatively and reduced mycoplasma binding by approximately 52%. No Fn binding inhibition was observed with pre-bleed sera at the same dilutions.

### Discussion

We screened M. pneumoniae for expression of FnBPs by SDS-PAGE ligand immunoblotting and Fn affinity column chromatography. These methodologies select for FnBPs that withstand harsh experimental conditions, such as detergent (SDS), temperature denaturation (100° C.), and reduction of disulfide bonds (2-ME). After such treatment, interactions observed between mycoplasma proteins and Fn would likely involve linear and not conformation-dependent domains. Using these screening conditions, we detected two M. pneumoniae FnBPs of sizes 45- and 30-kDa. Methodologies known to those skilled in the art employing least harsh conditions can be used to determine if conformation-dependent domains of intracellular enzymes bind fibronectin or other cell surface moleucules.

The identification of EF-Tu and PDH-B as the 45- and 30-kDa FnBPs, respectively, was accomplished by microsequencing and facilitated by the recent report delineating the entire M. pneumoniae genome (Himmelreich et al., Nucleic Acids Res, 24:4420-4449, 1996). The size of the predicted molecular weight of EF-Tu (43-kDa) matched with the size of the FnBP observed by ligand immunoblotting and affinity column chromatography. The slight discrepancy between the predicted molecular weight of the mature PDH-B protein (36-kDa) and that observed (30 to 35-kDa) is unclear although aberrant migration has been observed with proteins containing relatively large numbers of charged amino acids (Alderuccio et al., J Exp Med, 173: 941-52, 1991). The Fn binding activities attributed to EF-Tu and PDH-B were affirmed by the expression of these genes in E. coli as His¹⁰ tagged fusion proteins. These recombinant fusion proteins bound Fn using the immunoligand blot assay and dose dependent saturable ELISA.

The Fn-binding properties of ‘cytoplasmic’ proteins, EF-Tu and PDH-B, were unexpected. EF-Tu, which is responsible for critical steps in protein synthesis, has never been reported to exhibit Fn-binding activity or binding to any ECM protein or other host cell surface molecule. The observed Fn-binding activities of M. pneumoniae EF-Tu and PDH-B suggested a possible surface location. Using WCRIP we demonstrated the surface location of EF-Tu and PDH-B, which suggested that translocation and surface-accessible membrane conformation of these proteins could mediate Fn binding, which in turn could facilitate mycoplasma colonization of host tissues. Consistent with this scenario we demonstrated that [³⁵S]-methionine biosynthetically labeled viable M. pneumoniae cells adhere to immobilized Fn in a dose-dependent manner, and this binding was markedly reduced by pretreatment of mycoplasmas with anti-rEF-Tu and anti-rNPDH antisera. Additional M. pneumoniae FnBPs may exist which require tertiary conformation and may explain the residual Fn binding activities observed (FIG. 8). Using the techniques outlined in this specification as well as other techniques known to those skilled in the art, other such FnBPs can be identified. Additionally, binding to other molecules on the surface of a host cell can be determined for mycoplasma and other microorganisms. Also, the possibility exists that detailed identification of Fn-binding epitopes on M. pneumoniae EF-TU and PDH-B will further maximize Fn blocking effects. See Example 3 for some strategies for achieving such results. Additional strategies and methodologies known by those of skill in the art can also be used.

We propose that the surface membrane location of EF-Tu and PDH-B in M. pneumoniae provides additional and unique mechanisms by which mycoplasmas colonize tissues and gain intracellular residence. A range of intrinsic factors, such as tissue microenvironment, nutrient deprivation, immune surveillance, metabolic state of host and pathogen, and other stress or physiological cues, could signal the translocation of a subpopulation of cytoplasmic proteins to the microbial membrane surface. Under these situations conformational changes and alternative functions of ‘cytoplasmic’ molecules translocated to the membrane could occur. Overall, these observations suggest that the translocation of EF-Tu and PDH-B, and possibly other ‘cytoplasmic’ molecules, to the M. pneumoniae surface represents an important and deliberate biological and regulatory event that increases phenotypic versatility of pathogenic mycoplasmas by maximizing their limited genomic capabilities. Furthermore, we suggest that this membrane surface conformation of EF-Tu and PDH-B not only confers new biological functions but also identifies novel vaccine candidates and targets for anti-infective therapies.

### EXAMPLE 2

### Mycoplasma genitalium Adheres Selectively to Mucin and Antibodies to Translocated Molecules Inhibit this Binding

In order to identify targets of extracellular adherence to M. genitalium, we examined M. genitalium interactions with the primary components of the mucosal epithelial lining. The mucosal epithelium is coated with a thick gelatinous layer of glycoproteins, of which the primary component is mucin. The ability of pathogenic microorganisms to bind mucin and its correlation with virulence have been well-documented in uropathogenic Escherichia coli, Helicobacter pylori, Pseudomonas aeruginosa, and Haemophilus influenza. The interaction between mucin and M. genitalium has not been studied. Here we report that a translocated surface-expressed protein of M. genitalium binds specifically and in a concentration-dependent manner to mucin. We also report that antibodies to GAPDH inhibit adherence of M. genitalium to mucin, suggesting a potential therapeutic role for such antibodies and novel vaccine candidates and targets for anti-infective therapies.

### Materials and Methods

Mycoplasma Cultures

Wild-type M. genitalium G37 human isolate was grown in SP-4 medium at 37° C. for 72 hours in 150-cm² culture flasks. Surface-adherent mycoplasmas were harvested by washing 3 times with phosphate buffered saline (PBS) [150 mM NaCl, 10 mM sodium phosphate, pH 7.4] and collected by centrifugation at 12,500×g for 15 min at 4° C.

Bacterial Strains

Escherichia coli INVαF′[F′ endA1 rec1hsdR17spe44gyrA96lacZM15(lacZYA argG).] (Invitrogen, Carlsbad, Calif.) and E. coli BL2 (DE3) [F′ompT hsdSB (rB- mB-) gal dcm (DE3) pLysS] (Stdier) were grown in Luria Bertani (LB) broth and used to clone and express the M. genitalium GAPDH gene. For DNA manipulations the following vectors were used: pCR2.1 (AmprKmr TA cloning vector [Invitrogen]) and pET 16b (Apr,N-terminal His tag, expression vector [Novagen, Madison, Wis.]).

Radiolabelling of M. genitalium

M. genitalium was grown in SP-broth to logarithmic phase and harvested as described earlier. Mycloplasmas were resuspended (1/10^{th} of the original volume) in DMEM without cystiene or methionine and supplemented with 10% FBS. One mCi ³⁵S-methionine was added, and mycoplasmas were incubated at 37° C. for 4 hours.

Mucin Isolation

Cerival and vaginal mucin was collected as described in Venegas et al., Infect Immun., 63(2):416-22, 1995 and combined. Briefly, mucin was obtained from human patients using a sterile tongue depressor, which was placed in 3 ml of sterile PBS (pH 7.3). Cellular debris was pelleted by centrifugation at 9,000×g. Supernatant fractions, which contained mucin, were collected and partially purified by size exclusion column chromatography with G200 pore Sepharose. Mucin in the void volume was concentrated using Centricon 30 spin column (Amicon).

Mucin Binding Assays

Microtiter wells were coated with cervical/vaginal or bovine submaxillary type I (BSI, Sigma) mucin in concentrations ranging from 0.02 to 20 mg in 50 μl volumes. Unoccupied sites were blocked with 200 μl of 1 mg/ml BSA in PBS containing 0.05% Tween-20 (PBST). Wells coated with BSA alone served as negative controls. ³⁵S-Met biosynthetically labeled, viable M. genitalium G37 cells (100 μl, 10⁷ cells/well) were added to the coated wells for one hour, prior to rinsing microtiter plates with PBS buffer 3 times. Microtitre wells were detached and dissolved, along with radiolabeled bacterial cells, in scintillation fluid and radioactivity measured. Competitive inhibition of binding was monitored by preincubating radiolabeled mycoplasmas with 2 μg of mucin prior to their addition to mucin-coated plates and by preincubating radiolabeled mucoplasmas with 10, 50, and 100 mM quantities of mucin-associated sugars (fucose, sialic acid, N-acetylglucosamine, N-acetylgalactosamine, and galactose). Rhamnose and mannose were used as negative controls.

DNA Extraction

M. genetalium DNA was prepared as described in Reddy et al., J. Bacteriol., 177(20):5943-51, 1995. Plasmid DNA was purified using the QIAprep spin protocol as described by the manufacturer (Quiagen).

Column Chromatography

Mucin-coupled epoxy agarose columns were prepared as described by the manufacturer (Pharmacia) with the following exceptions. Three ml volumes of epoxy slurry (swelled) were coupled to 1.2 mg of mucin in 2 ml (615 μg.ml) of coupling buffer (borate buffer 0.05 M, pH 9.0) at 25C for 16 h. Completion of coupling was determined by measuring optical displacement at 260 nm (OD260). Excess uncoupled epoxy groups were blocked with 1 M ehtanolamine overnight. An optical displacement of 0.0 was indicative of complete binding, althoughm the lowest OD260 obtained was 0.26. To identify mycoplasma proteins responsible for adherence to mucin, we pre-adsorbed M. genitalium G37 total solubilized proteins with uncoupled epoxy agarose resin prior to performing mucin-epoxy agarose resin chromatography. Mucin-binding proteins were eluted by adding 2.5 M LiCl. Approximately 18 fractions of 1 ml each were collected and the OD260 of each fraction measured. Each fraction exhibiting a significant increase in OD was concentrated in a Centricon column, separated by SDS-PAGE, and transferred to PVDF membrane for microsequencing.

N-Terminal Protein Sequencing

PVDF blots of SDS-acrylamide gels containing MnBPs were stained with 0.1% Ponceau S solution (w/v) and washed thoroughly in distilled water. Individual protein bands were excised from the blot and subjected to Edman degradation sequencing by the microsequencing facility at Baylor College of Medicine (Houston, Tex.).

Cloning of GAPDH

Based on the published genome of M. genitalium, the complete open reading frame of GAPDH was amplified using the 5′ primer, 5′-CTAATTATTAAATTAACATATGGCAGCAAG-3′ (SEQ ID NO: 7), and the 3′ primer, 5′-TAACCCCATGGATCCTTGGGACATTAA-3′ (SEQ ID NO: 8), producing BamHI and NdeI restriction sites (bold) at the 5′ and 3′ ends of GAPDH, respectively. The fragment was ligated into pCR 2.1 vector and transformed into E. coli INVαF′ cells. The resultant plasmid was designated pCR-GAPDH.

Expression and Purification of Recombinant Proteins

DNA fragments generated by NdeI and BamHI digestion of plasmid pCR-GAPDH were ligated into the pET-16B expression vector. These plasmids were transformed into competent E. coli BL21 (DE3) cells grown to a density of 2×10⁹ cells/ml at 37° C. in standard LB broth (Sigma) containing 100 μg/ml ampicillin. Induction of recombinant protein synthesis was accomplished by the addition of 100 μM of IPTG, and E. coli cultures were incubated further for 3 h at 37° C. under aerations at 220 rpm. Bacteria from 1 ml samples were pelleted, suspended in 250 μl of sample buffer, and heated to 95° C. for 5 min. 10 μl aliquots of test samples were analyzed on 10% SDS/polyacrylamide gels. Recombinant colonies were screened for resistance to ampicillin and for expression of a protein product of the correct size. One recombinant clone from each construct was selected for further study. Verification of specific clones was achieved by restriction digestion. Fusion proteins were purified from urea lysates of recombinant E. coli by nickel chromatography using the manufacturer's denaturing protocol (Quiagen)

Antibody Reagents

Rabbit monospecific antibody reagents were generated by The University of Texas Health Science Center Institutional Immunology Facility as described by Dallo et al., Infect. Immun., 2595-2601, 1996. New Zealand White rabbits were immunized with 2 mg of recombinant glyceraldehyde 3-phosphate dehydrogenase (RGAPDH) emulsified in Freund's complete adjuvant. On days 24, 43, and 59, rabbits received additional subcutaneous immunizations in Freund's incomplete adjuvant. Sera were tested for reactivity on whole M. genitalium proteins and rGAPDH.

Antibody Blocking Assays

³⁵S-Met biosynthetically labeled and viable mycoplasmas were pretreated with heat-inactivated anti-rGAPDH polyclonal serum, prebleed serum, or anti-P140 (MG191) and anti-P32 M. genitalium adhesin polyclonal sera at 1/1000 dillutions for 1 hour at 37° C. before incubating with immobilized mucin on ELISA plates as described earlier. Plates were rinsed with PBS, and radioactive counts were measured.

Whole Cell Radioimmunoprecipitation (WCRIP) Assay

To determine if the mucin-binding GAPDH protein of M. genitalium was surface accessible, we performed WCRIP as described by Dallo et al., Infect. Immun., 2595-2601, 1996. Briefly, ³⁵S-Met biosynthetically labeled mycoplasmas were pretreated with heat-inactivated anti-rGAPDH polyclonal serum (1/1000 dilution) or preimmune serum for 1 hr at 37° C. Cells were lysed, and M. genitalium immune complexes were precipated with protein A. Intact, viable mycoplasmas were also treated with trypsin prior to immunoprecipitation.

Mycoplasma Membrane Purification’

Mycoplasma membranes were isolated by osmotic lyses as described in Methods in Mycoplasmology. Briefly, mycoplasmas were harvested by centrifugation at 12,000×g for 15 min and washed in 0.25 M NaCl prior to resuspension in 2 ml of 0.25 M NaCl. Cells were then lysed by rapid transferring into 50-100 volumes of high-quality deionized water preheated to 37° C. Samples were incubated at this temperature for 15 min, and membranes were collected by centrifugation at 34,000×g for 30 min. Membranes were washed sequentially in deionized water, 0.05 M NaCl in 0.01 M phosphate buffer, pH 7.5, and deionized water.

Immunogold Electron Microscopy

Fresh intact M. genitalium cells were washed in PBS (pH 7.5) and incubated with PBS (pH 7.5) containing 0.1% gelatin:type B from Bovine Skin (Sigma) to reduce non-specific binding. For single gold particle labeling, cells were incubated 2 hr at 37° C. with anti-rGAPDH sera diluted (1:100) in PBS w/gelatin:type B (0.1%). Mycoplasmas were then washed with PBS three times and incubated for 60 min at room temperature with goat anti-rabbit immunoglobulin G (IgG)-gold complex (average particle size, 10 nm, 1:20 dilution) suspended in PBS (PH 7.5). After washing three times with PBS, mycoplasmas were mounted onto Formvar-coated nickel grids by fixing with 1% glutaraldehyde-4% formaldehyde for 20 min at room temperature. Finally, grids were stained with 7% uranyl acetate followed by Reynolds lead citrate and examined with a Philips 208S Transmission Electron Microscope at ˜60 kv accelerating voltage.

GAPDH Enzymatic Activity

To confirm that recombinant GAPDH was in its native functional form and to further implicate its membrane localization, we measured the enzymatic activity of GAPDH using the Ferdinand GADPH enzymatic assay (Ferdinand, W., Biochem J., 92(3):578-85, 1964). Mycoplasma rGAPDh, yeast GAPDH (Sigma), and M. genitalium purified membranes were diluted to a concentration of 10 μg/ml in EDTA at pH 8.6. 100 μl amounts of 10 mM NAD+ and 20 mM DL-glyceraldehyde phosphate substrate were combined with 50 μl of test preparation plus 750 μl of reaction buffer (10 mM ethanolamine, 20 mM Na₂PO₄, 50 mM EDTA). Optical density at 360 nm was measured over a 4 min time span using a Beckman 530 spectrophotometer.

### Results

Mucin Binding

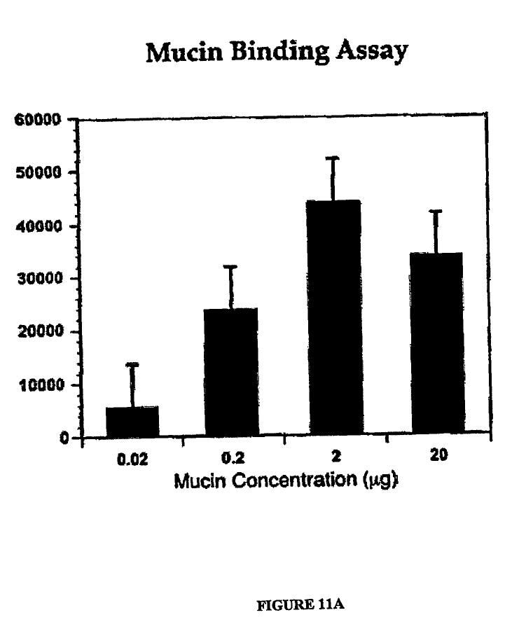

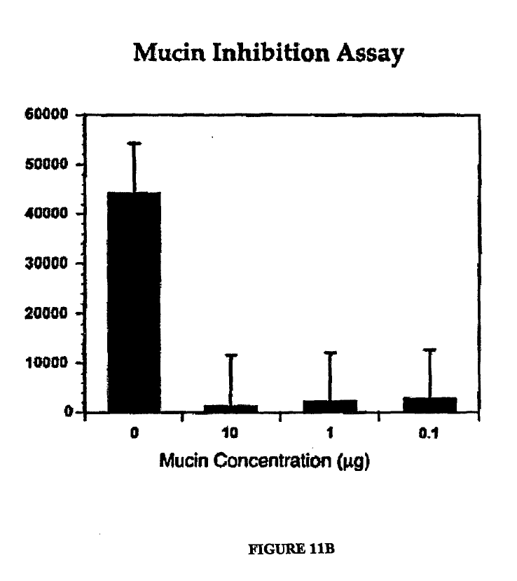

M. genitalium whole cells biosynthetically radiolabeled with ³⁵S Met bound to mucin in a dose dependent manner (FIG. 11A). To further determine M. genitalium binding specificity to mucin, we preincubated radiolabeled mycoplasmas with 0 to 10 μg of mucin or albumin prior to their addition to mucin-coated microtitre wells. M. genitalium binding to mucin was inhibited greater than 95% following mucin pretreatment (FIG. 11B), whereas albumin pretreatment was without effect. Furthermore, we determined whether binding to mucin was mediated by sugars linked to the mucin apoprotein. Radiolabeled mycoplasmas were preincubated with 50 mM quantities of the five mucin-associated sugars (fucose, galactose, sialic acid, N-acetylglucosamine, N-acetylgalactoseamine) as well as mannose and rhamnose, which served as negative controls. binding was inhibited by at least 70% with each mucin sugar, and much less by rhamnose and mannose (FIG. 11C).

Isolation and Identification of Mucin Binding Proteins (MnBPs)

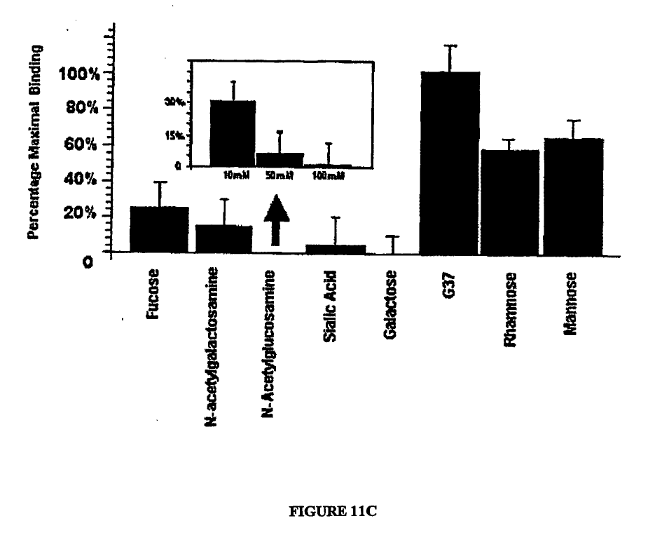

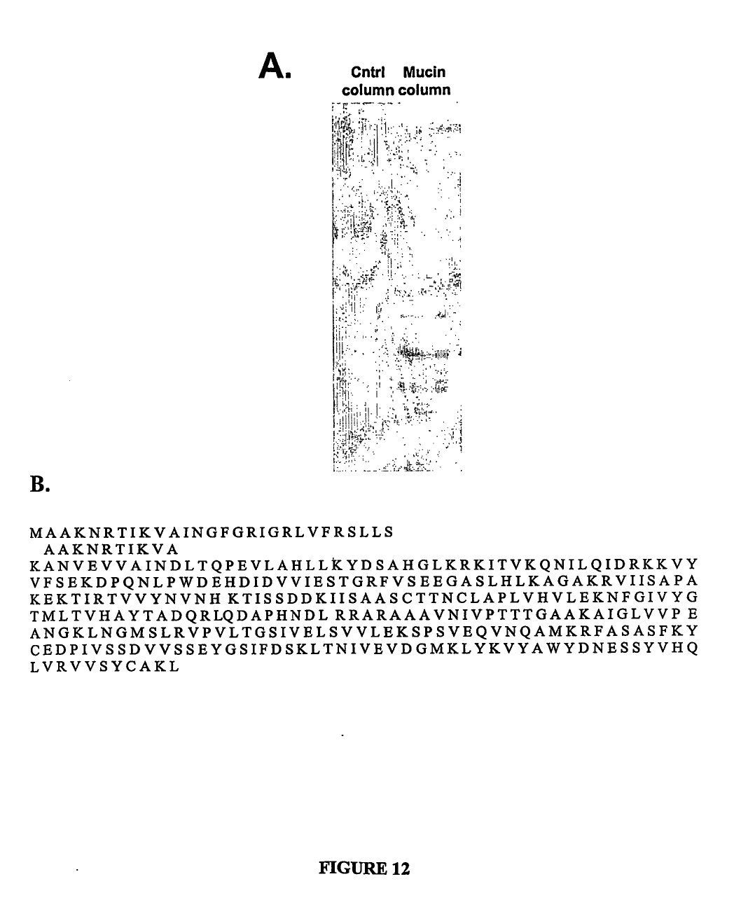

To determine which M. genitalium proteins were responsible for mucin binding, we first preadsorbed ³⁵S Met-labeled total M. genitalium protein lysates with uncoupled epoxy agarose resin to minimize non-specific binding followed by mucin-epoxy affinity column chromatography. Three mycoplasma proteins of 36, 38 and 40 kDa were eluted with 2.5 M LiCl (FIG. 12A). The purified 38 kDa protein was N-terminal sequenced and identified as glyceraldehyde 3-phosphate dehydrogenase (GAPDH) (FIG. 12B). The other proteins were also sequenced and were shown to be pyruvate dehydrogenase subunits A and B.

Characterization of Recombinant GAPDH (rGAPDH)

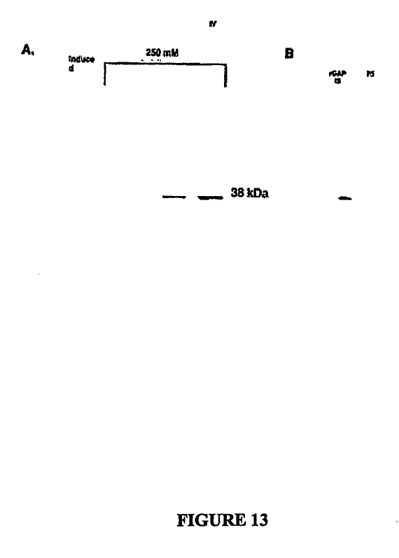

rGAPDH was expressed using the pET16b vector in E. coli and purified by ion-exchange chromatography (FIG. 13A). The purified protein was used to generate specific rabbit polyclonal serum, which was confirmed by immunoblot (FIG. 13B). To further establish the functional properties of rGAPDH, we measured GAPDH enzymatic activity based on NADH oxidation. Both rGAPDH and purified mucoplasma membranes oxidized NADH with O.D. 340 nm values reaching 0.06 and 0.05 during a 1.5 min interval, respectively. During the same period yeast GAPDH reached an O.D. value of 0.03 where as the blank O.D. remained 0.

Surface Accessible Location of M. genitalium GAPDH

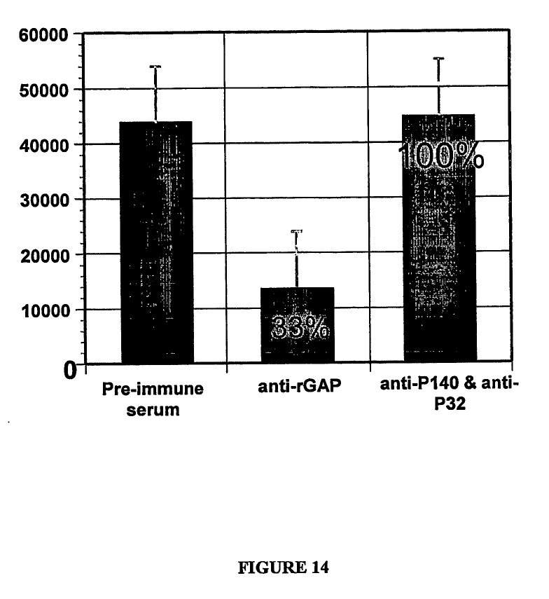

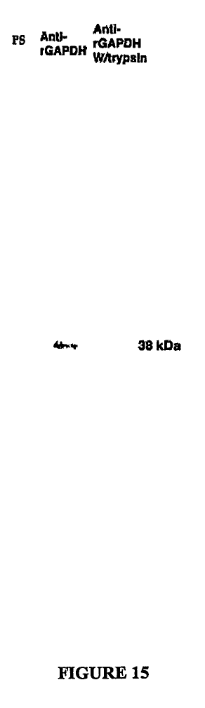

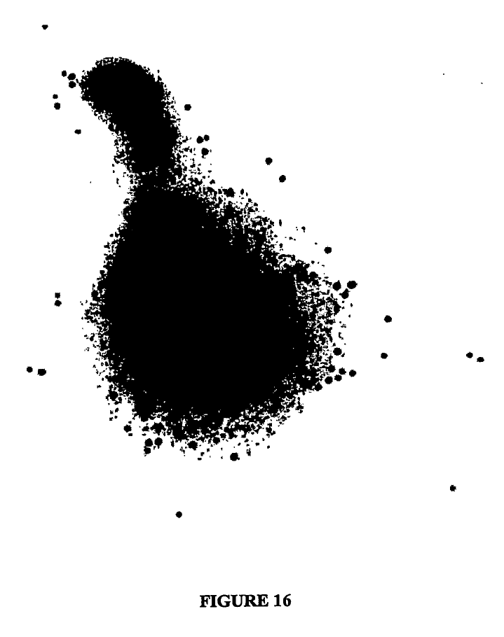

The mucin-binding property of GAPDH and its enzymatic activity in mycoplasma membranes suggested that GAPDH might in part mediate M. genitalium binding to Mn (FIG. 11) and therefore be surface exposed. To establish the surface location of GAPDH we first performed blocking assays using antisera generated against rGAPDH. Radiolabeled mycoplasmas were pretreated with anti-rGAPDH serum, which reduced mycoplasma binding to mucin by 67% (FIG. 14). Pre-immune serum and antiserum generated against the P140 and P32 tip-associated adhesions of M. genitalium had no effect. We also performed whole cell radioimmunoprecipitation (WCRIP) using anti-rGAPDH antiserum to further establish surface accessibility of GAPDH. Aliquots of radiolabeled mycoplasmas were incubated either with heat-inactivated monospecific anti-rGAPDH or preimmune rabbit sera. In addition, test samples pretreated with trypsin to identify protease-sensitive surface proteins. As presented in FIG. 13, antiserum against rGAPDH recognized a 38-kDa surface-associated protein, while preimmune serum was nonreactive. Additionally, the 38-kDa protein was absent when intact mycoplasmas were treated with trypsin prior to WCRIP, further establishing the surface-exposed conformation of GAPDH (FIG. 15). Furthermore, in order to more conclusively demonstrate the presence of GAP on the surface of M. genitalium, immunoelectrom microscopy with antiserum to rGAP was performed. Immune serum labeled cells demonstrates the presence of immunogold labeled antibody on the surface of intact whole M. genitalium cells (FIG. 16).

Pyruvate Dehydrogenase

Using similar techniques additional intracellular enzymes of M. genitalium that bind mucin were identified, namely, pyruvate dehydrogenase E1 alpha subunit (PDH-A) and pyruvate dehydrogenase E1 beta subunit (PDH-B).

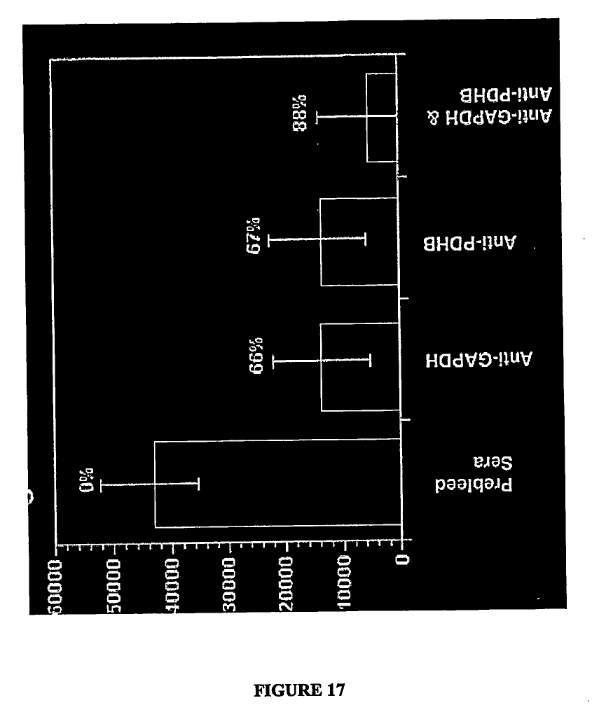

An additional study was done to determine the effect of antibodies to GAPDH and PDHB on binding of M. genitalium to mucin. The results are shown in FIG. 17. Antibodies to GAPDH inhibited mycoplasma binding to mucin by 66%, antibodies to PDHB inhibited mycoplasma binding to mucin by 67%, and antibodies to both GAPDH and PDHB inhibited binding by 88%.

### Discussion

M. genitalium binding to mucin was dose dependent and inhibited by preincubation with mucin. Microsequencing data indicated that one of the mucin-affinity purified mycoplasma proteins was the metabolic enzyme, glyceraldehyde 3-phosphate dehydrogenase. Polyclonal antibody raised against the recombinant protein inhibited the binding of viable M. genitalium to mucin by 70%. Furthermore, whole cell radioimmunoprecipitation with anti-GAPDH polyclonal antibodies demonstrated that the protein is surface localized. These data indicate that not only does GAPDH bind to mucin, but also is translocated to the surface in a biologically relevant conformation.

### EXAMPLE 3

### Identification of Epitope(s) of Intracellular Enzymes Important for Binding the Surface of Host Cells and Generation of Antibodies Thereto

Epitopes of translocated enzymes of microorganisms important for binding the surface of host cells or molecules thereof can be readily identified using the methodologies and strategies described herein. Other known techniques are readily available and can also be used to identify epitopes of translocated enzymes involved in binding the surface of a host cell or molecules thereof.

For example, translocated enzymes of microorganisms can be identified as described in Examples 1 and 2. Once a terminal amino acid sequence is obtained, public and commercial databases can be searched with the sequence to obtain fill length or partial sequences of the enzyme. Truncated fragments of translocated enzymes can be expressed in a variety of known expression hosts through use of known recombinant DNA techniques. The truncated fragment can be expressed and purified from the host microorganism using known techniques.

The truncated fragment can be expressed on the surface of the expression host by expressing the fragment as a fusion peptide with a peptide known to be expressed on the surface of the expression host. The truncated fragment can be expressed as a fusion with a known tag peptide that can be purified by known methods. Preferably, the fusion peptide can be cleaved to release the truncated fragment using known techniques. Other known methods for linking and separating the truncated fragment from the peptide can be employed.

The ability of a truncated fragment to bind the surface of a host cell or molecules thereof can be determined as described in Examples 1 and 2. Binding of one or more truncated fragment to one or more extracellular matrix molecule or fragments thereof can be determined using the truncated fragment, the truncated fragment linked to the peptide, or the truncated fragment expressed at the surface of the host cell. Binding affinities of the one or more fragment to the one or more extracellular molecule or a fragment thereof can be determined by using known methodologies. Those fragments having strong binding affinities would be expected to be important for binding to the surface of the host cell.

One or more fragments determined to bind to the one or more extracellular matrix molecules can be used to generate antibodies described herein or using other known techniques. The antibodies can be used in an assay to determine if they are capable of inhibiting the binding of the microorganism to either the surface of a host cell or to an artificial extracellular matrix.

### EXAMPLE 4

### Mapping of Mucin Binding Domains in Glyceraldehyde 3-phosphate Dehydrogenase

The GAPDH gene (MG301) was truncated at the carboxy terminus by subsequent 30 amino acid deletions. The amino acid sequence of the GAPDH gene (MG301) is:

| | |
|---|---|
| MAAKNRTIKV AINGFGRIGR LVFRSLLSKA | (SEQ ID NO: 9) |
| NVEVVAINDL TQPEVLAHLL KYDSAHGELK | |
| RKITVKQNIL QIDRKKVYVF SEKDPQNLPW | |
| DEHDIDVVIE STGRFVSEEG ASLHLKAGAK | |
| RVIISAPAKE KTIRTVVYNV NHKTISSDDK | |
| IISAASCTTN CLAPLVHVLE KNFGIVYGTM | |
| LTVHAYTADQ RLQDAPHNDL RRARAAAVNI | |
| VPTTTGAAKA IGLVVPEANG KLNGMSLRVP | |
| VLTGSIVELS VVLEKSPSVE QVNQAMKRFA | |
| SASFKYCEDP IVSSDVVSSE YGSIFDSKLT | |
| NIVEVDGMKL YKVYAWYDNE SSYVHQLVRV | |
| VSYCAKL. | |

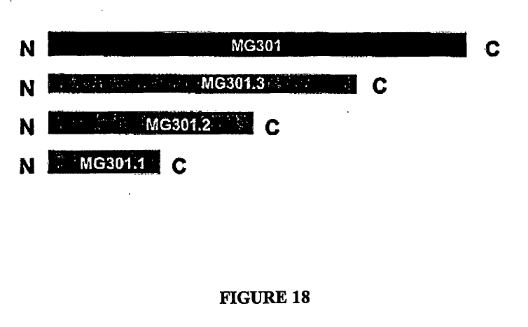

A schematic representation of the truncated fragments is presented in FIG. 18. Each peptide fragment, including the complete GAPDH, was expressed in the pET16B expression vector and each fragment was purified by nickel column chromatography. The ability of the truncated proteins to bind mucin was then determined. All three fragments retained the ability to bind mucin at levels near binding levels of the complete GAPDH, indicating that the first 30 amino acids of the protein are important for binding. Therefore, the first 30 amino acids may serve as an effective vaccine candidate, diagnostic tool, and/or target for anti-microbials.

Accordingly, an epitope of the invention can comprise at least a portion of the first 30 amino acids of GAPDH: MAAKNRTIKV AINGFGRIGR LVFRSLLSKA (SEQ ID NO: 10). An epitope having at least 80%, 85%, 90%, 95%, or 99% sequence identity with SEQ ID NO:10 may also be suitable for the present invention. An immunizing composition comprising a peptide including at least a portion of SEQ ID NO: 10 or a peptide having at least 80%, 85%, 90%, 95%, or 99% sequence identity with SEQ ID NO:10 may also be suitable for the present invention. In addition, molecules which inhibit binding of a peptide comprising at least a portion of SEQ ID NO: 10 or a peptide having at least 80%, 85%, 90%, 95%, or 99% sequence identity with SEQ ID NO:10 to the surface of a host cell may be suitable for the present invention.

### EXAMPLE 5

### Binding of M. genitalium to Mucin-Coated Surfaces.

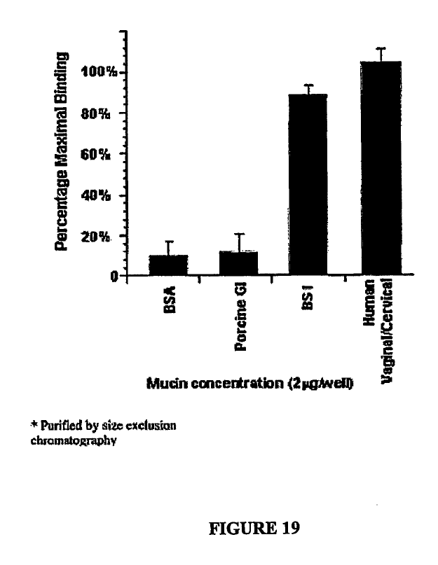

Plates were coated with 2 μg of human vaginal/cervical (V/C) mucin, porcine gastrointestinal (GI) mucin, bovine submaxillary mucin (BSI) and bovine serum albumin (BSA). Radiolabeled mycoplasmas were then added to each well. Plates were rinsed with PBS and radioactivity determined. The results, which are presented in FIG. 19, indicate that M. genitalium binds to mucin from all the above-mentioned sources. M. genitalium bound with highest affinity to human vaginal/cervical mucin. High binding was also observed with bovine submaxillary mucin.

### EXAMPLE 6

### Binding of M. pneumoniae to Fibronectin is Inhibited by EF-Tu, PDH-B and EF-Tu and PDH-B.

Microtitre plate wells were coated with 100 ng of Fn for 16 h at 4° C. The Fn coated wells were then preincubated with 100 μg (2×), 50 μg (1×), 25 μg (0.5×), or 12.5 (0.25×) μg recombinant EF-Tu, 150 μg (2×), 75 μg (1×), 37.5 μg (0.5×), or 18.75 μg (0.25×) recombinant PDH-B and 1×EF-Tu/1×PDH-B in combination at 37° C. for 2 h. Individual wells with Fn and M. pneumoniae served as positive control. Individual wells with Fn and M. pneumoniae served as positive control. Individual wells with BSA and M. pneumoniae served as negative control.

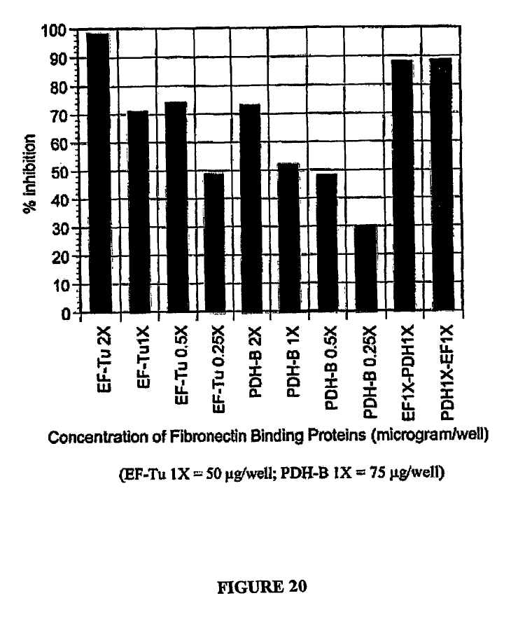

As shown in FIG. 20, preincubation with all tested concentrations of EF-Tu, PDH-B and EF-Tu in combination with PDH-B inhibited binding of M. pneumoniae to Fn. Nearly 100% of binding was inhibited by preincubation with EF-Tu at 2×, and nearly 90% inhibition was observed with the combination of 1×EF-Tu with 1×PDH-B.

### EXAMPLE 7

### Transmembrane Domains of EF-Tu and PDH-B

Transmembrane helices in integral membrane proteins are composed of stretches of 10-30 predominantly hydrophobic residues separated by polar connecting loops. A number of algorithms designed to identify putative transmembrane helices in the primary amino acid sequence have been developed, and current methods can identify around 90-95% of all true transmembrane segments with an over-prediction rate of only a few percent (von Heijne, J. Mol. Biol., 225:487-494, 1992; Rost et al., Protein Sci., 5:1074-1718, 1996).

Dense Alignment Surface (DAS) method was introduced in an attempt to improve sequence alignments in the G-protein coupled receptor family of transmembrane proteins (Cserzo et al., J Mol. Biol., 243:388-396, 1994) and now extended to predict transmembrane segments in any integral membrane protein. DAS is based on low-stringency dot-plots of the query sequence against a collection of non-homologous membrane proteins using a previously derived, special scoring matrix and is more specific for bacterial proteins.

Using the DAS program, two potential transmembrane domains in the amino acid sequence of EF-Tu and one in PDH-B were identified, supporting their possible surface localization. Those portions of EF-Tu and PDH-B which would be exposed to the extracellular environment of a mycoplasma would be likely therapeutic and/or diagnostic candidates in accordance with the present invention.

1. A method for inhibiting binding of a microorganism to a surface of a host cell, comprising:
contacting the microorganism or the surface of the host cell with one or more inhibiting molecules that interacts with
a) one or more translocated molecules of the microorganism, or
b) one or more surface molecules of the host cell, or
c) one or more translocated molecules of the microorganism and one or more surface molecules of the host cell,
wherein the one or more inhibiting molecules inhibits binding between the surface of the host cell and the one or more translocated molecules,
with the proviso that the one or more translocated molecules is not GAPDH.

2. The method of claim 1, wherein the translocated molecule is a non-glycolytic enzyme or a portion thereof.

3. The method of claim 1, wherein the translocated mole is an anabolic enzyme or a portion thereof.

4. The method of claim 1, wherein the translocated molecule is EF-Tu or a portion thereof.

5. The method of claim 1, wherein the translocated molecule is pyruvate dehydrogenase or a portion thereof.

6. The method of claim 5, wherein the translocated molecule is PDH-B or a portion thereof.

7. The method of claim 5, wherein the translocated molecule is PDH-A or a portion thereof.

8. The method of claim 1, wherein the one or more surface molecule of the host cell comprises one or more extracellular matrix proteins.

9. The method of claim 8, wherein the one or more extracellular matrix protein comprises mucin.

10. The method of claim 8, wherein the one or more extracellular matrix protein comprises fibronectin.

11. The method of claim 1, wherein the microorganism is a mycoplasma.

12. The method of claim 11, wherein the mycoplasma is Mycoplasma pneumoniae.

13. The method of claim 11, wherein the mycoplasma is Mycoplasma genitalium.

14. The method of claim 1, wherein the one or more inhibiting molecules are one or more antibodies to one or more translocated molecules of the microorganism.

15. The method of claim 14, wherein the one or more antibodies are directed to an epitope of a translocated molecule involved in binding the surface of the host cell.

16. The method of claim 1, wherein the one or more inhibiting molecules comprise a mucin-associated sugar.

17. The method of claim 16, wherein the mucin-associated sugar is selected from the group consisting of fucose, N-acetylgalactosamine, N-acetylglucosamine, sialic acid, and galactose, or a combination thereof.

18. The method of claim 1, wherein the one or more inhibiting molecules comprise the translocated molecule.

19. The method of claim 18, wherein the translocated molecule is a species-specific homolog of the translocated molecule.

20. A method for inhibiting binding of a mycoplasma to a surface of a host cell, comprising:
contacting the mycoplasma or the surface of the host cell with one or more inhibiting molecules that interacts with
a) one or more translocated molecules of the microorganism, or
b) one or more surface molecules of the host cell, or
c) one or more translocated molecules of the microorganism and one or more surface molecules of the host cell,
wherein the one or more inhibiting molecules inhibits binding between the surface of the host cell and the one or more translocated molecules.

21. A method for inhibiting binding of a microorganism to a surface of a host cell, comprising:
contacting the microorganism or the surface of the host cell with one or more inhibiting molecules that interacts with
a) one or more translocated molecules of the microorganism, or
b) one or more surface molecules of the host cell, or
c) one or more translocated molecules of the microorganism and one or more surface molecules of the host cell,
wherein the one or more inhibiting molecules inhibits binding between the surface of the host cell and the one or more translocated molecules, and one or more of the translocated molecules binds mucin.

22. An isolated epitope of a translocated molecule of a microorganism, wherein the epitope is involved in binding the microorganism to a surface of a host cell.

23. The isolated epitope of claim 22, wherein the epitope is linked to a carrier.

24. The isolated epitope of claim 22, wherein the epitope comprises a peptide comprising at least a portion of the following amino acid sequence: MAAKNRTIKV AINGFGRIGR LVFRSLLSKA (SEQ ID NO:10).

25. An antibody to the isolated epitope of claim 22.

26. A composition comprising the antibody of claim 25 in a pharmaceutically acceptable carrier.

27. A composition comprising the isolated epitope of claim 22 in a pharmaceutically acceptable carrier.

28. The composition of claim 27 further comprising an adjuvant.

29. The composition of claim 27, wherein the isolated epitope is linked to a carrier.

30. A method for treating a a subject for an infection caused by a microorganism comprising:
administering to the subject one or more antibodies to one or more translocated molecules of the microorganism,
wherein the one or more antibodies inhibits binding between the surface of the host cell and the one or more translocated molecules.

31. The method of claim 30, wherein the subject is human.

32. A method for treating a subject for an infection caused by a microorganism comprising:
administering to the subject one or more antigens of one or more translocated molecules of the microorganism;
wherein a humoral response to the antigen is produced, thereby producing one or more antibodies to the one or more translocated molecules, and
wherein the one or more antibodies inhibits binding between the surface of the host cell and the one or more translocated molecules.

33. The method of claim 32, wherein the subject is human.

34-41. (canceled)
